(19) United States
(12) Patent Application Publication
(10) Pub. No.: US 2019/0127381 A1
(43) Pub. Date: May 2, 2019

(54) SUBSTITUTED FUSED PYRIMIDINONE COMPOUNDS

(71) Applicant: TORRENT PHARMACEUTICALS LIMITED, Ahmedabad (IN)

(72) Inventors: Chaitanya DUTT, Gandhinagar (IN); Deepa JOSHI, Gandhinagar (IN); Milind RODE, Gandhinagar (IN); Sanjay SRIVASTAVA, Gandhinagar (IN); Davinder TULI, Gandhinagar (IN); Ramesh Chandra GUPTA, Gandhinagar (IN); Deepak RAI, Gandhinagar (IN); Prashant JAMADARKHANA, Gandhinagar (IN); Shailesh DESHPANDE, Gandhinagar (IN); Vivek MISHRA, Gandhinagar (IN); Jaya ABRAHAM, Gandhinagar (IN)

(73) Assignee: TORRENT PHARMACEUTICALS LIMITED, Ahmedabad (IN)

(21) Appl. No.: 16/095,597

(22) PCT Filed: Apr. 25, 2017
(86) PCT No.: PCT/IB2017/052352

§ 371 Date: Oct. 22, 2018

(30) Foreign Application Priority Data
Apr. 26, 2016 (IN) 201621014526

(51) Int. Cl. C07D 487/04; A61P 11/06

(52) U.S. Cl. CPC C07D 487/04; A61P 11/06

(57) ABSTRACT

Provided herein are novel fused pyrimidinone derivatives of Formula I and synthetic intermediates that are useful in preparing the compounds of formula (I). Further provided herein is a method for preparation of a compound of formula (I) or intermediates, a pharmaceutical composition comprising a compound of formula (I), and use of a compound of formula (I).

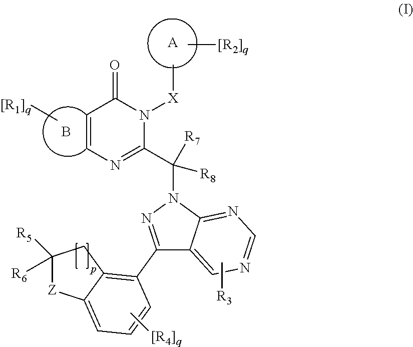

### CROSS-REFERENCE TO RELATED APPLICATION

This application is a National Stage filing under 35 U.S.C. § 371 of PCT/IB2017/052352 filed on 25 Apr. 2017, which claims priority to Indian Patent Application No. 201621014526 filed 26 Apr. 2016, entitled “Substituted Fused Pyrimidinone Compounds”.

### FIELD OF THE INVENTION

The present invention relates to fused pyrimidinone derivatives their pharmaceutically acceptable salts, and their metabolites, isomers, steroisomers, atropisomers, conformers, tautomers, polymorphs, hydrates and solvates. The present invention also encompasses pharmaceutically acceptable compositions of said compounds and process for preparing novel compounds. The invention further relates to the use of the above mentioned compounds for the preparation of medicament for use as pharmaceuticals.

### BACKGROUND

The prevalence of airway diseases has increased in recent decades despite therapeutic advances. Among the airway diseases, asthma exacerbations and chronic obstructive pulmonary disease (COPD) are major causes of hospitalization. Both asthma and COPD involve chronic inflammation of the respiratory tract. COPD is a term which refers to a large group of lung diseases characterized by obstruction of air flow that interferes with normal breathing. Emphysema and chronic bronchitis are the most important conditions that compose COPD. (Australian lung foundation, 2006) COPD involves chronic inflammation of the peripheral airways and lung parenchyma, which leads to progressive narrowing of the airways and shortness of breath. On the other hand, Asthma is characterized by episodic airway obstruction and symptoms and usually starts early in life. More recently it has become clear that severe asthma is much more similar to COPD, with similarities in the inflammation and sharing a poor response to corticosteroids (J. Allergy Clin Immunol. 2013; 131(3):636-45). Interestingly, studies of molecular genetics are now showing that severe asthma and COPD share several gene polymorphisms (Comp Funct Genomics. 2012; 2012: 968267).

COPD is a major global health problem that is becoming prevalent, particularly in developing countries. It is one of the most common diseases in the world, with a lifetime risk estimated to be as high as 25%, and now equally affects both men and women (Nature Reviews 2013; 12: 543-559).

Current forms of therapy for COPD provide only symptomatic relief and are relatively ineffective, as there are no drugs available that considerably reduce disease progression or mortality or have a substantial effect on exacerbations, which are one of the most common causes of hospital admissions.

Long acting bronchodilators are the mainstay of current COPD therapy. There have been several advances in the development of β2-adrenergic receptor agonists and muscarinic receptor antagonists that only need to be administered once a day. Moreover, long acting β2-adrenergic receptor agonists (LABAs) and long acting muscarinic acetylcholine receptor antagonists (LAMAs) have additive effects on bronchodilation and in the improvement of symptoms, which has led to the development of LABA-LAMA combination inhalers. However, although these drugs produce effective bronchodilation, they fail to treat the underlying inflammatory disease in patients with COPD.

Alternatively, or additional to bronchodilators, oral or inhaled corticosteroids could also be used as COPD therapy. But corticosteroids have limitations as long term oral corticosteroid therapy is not recommended and inhaled corticosteroids are known to be associated with increased risk of pneumonia in patients (www.bcguidelines.ca). Moreover, inhaled corticosteroids are found largely ineffective in significant number of COPD patients as an anti-inflammatory therapy in COPD (Ann Fam Med. 2006; 4(3): 253-62). Phosphodiesterase inhibitors (PDE-4 inhibitors) have recently been shown to document clinical efficacy in COPD, although their utility is hampered by class related side effects (International Journal of COPD 2007; 2(2): 121-129).

With better understanding of the pathophysiology of COPD and asthma disease process and recognition of inflammation as an important feature, it is anticipated that disease modifying therapy for COPD and asthma targeting underlying inflammation will prove effective, the way it has been successful in the treatment of other chronic inflammatory conditions like RA.

To address the unmet need of anti-inflammatory therapy for COPD and asthma, novel promising targets are in clinical development including inhibitors of p38 mitogen-activated protein (MAP) kinase, inhibitor of nuclear factor-κB kinase-2 (IKK2), inhibitors of PI3-Kinase and Spleen Tyrosine Kinase (SYK). Several other kinases and transcription factors which are modulators of inflammatory signal transduction pathways are also targets for novel drug development for asthma and COPD.

PI3K or PI3-Kinase is categorized into class I, class II, and class III according to its primary structure and substrate specificity, with class I being involved in cellular survival and differentiation (Nature Reviews Genetics 2006; 7: 606-619). Class I PI3K is divided into four subunits, α, β, δ and γ subunits (Nature Reviews, Molecular Cell Biology, 2012; 13: 195-203). Expression of the PI3K α and PI3K β isoforms is ubiquitous, while the expression pattern of PI3K δ and PI3K γ seems more restricted, with both isoforms found primarily in leukocytes (J. Med. Chem. 2012, 55, 8559-858).

Selective inhibition of PI3K δ, γ or β has each been shown to reduce the severity of inflammation in one or more models of autoimmune and respiratory diseases like COPD and asthma (Biochimica et Biophysica Acta 1851; 2015:882-897). The role of PI3K δ and γ in immunodeficiency and respiratory disease was further reinforced by the discovery of activated PI3K δ syndrome (APDS) as well as missense mutation of PI3K γ in humans characterized by recurrent respiratory infections, progressive airway damage, inflammation and cutaneous fungal infections (Science, 2013; 342(6160):866-871; PLoS One, 2013; 8(7): e68118).

PI3K δ and γ expressed in all the immune cells including Neutrophils, Macrophages, monocytes, Mast cells, Eosinophil, T and B cells, coordinating inflammation in COPD lungs during various stages of COPD (Ther Adv Resp Dis. 2010,3(1): 19-34). PI3K-δ and PI3K-γ inhibitors have been reported to suppress inflammation in animal model of COPD. The relatively restricted expression pattern of PI3K δ and PI3K γ, as well as data from mice, where PI3K δ and/or PI3K γ was either genetically or pharmacologically inactivated, suggests that these two isoforms play a major role in the adaptive and innate immune systems (J. Med. Chem. 2012, 55, 8559-8581). Though the selective PI3K γ and PI3K δ inhibitors are proven to inhibit the inflammatory signaling, recent findings suggests its interdependent and cooperative role. Combination of PI3K δ and PI3K γ inhibitors appears to be superior to selective inhibition of single isoform to curtail efficient trafficking of immune competent cells to sites of inflammation.

In spite of known targets and pathway of controlling inflammation, there has been no drug available till date to effectively treat the underlying mechanism of disease conditions including COPD and Asthma.

Some prior arts disclose the compounds as PI3K inhibitors, for example WO2009088990 and WO2009088986 discloses compounds that modulate PI3K activity. Similarly, WO2012037204 discloses PI3K delta inhibitors.

There remains a need to identify and develop new compounds, predominantly having selectivity for PI3K δ and γ, which provides desired therapeutic potential along with improved pharmacokinetic profile and/or lesser side effects. Present invention provides novel fused pyrimidinone derivatives as PI3K inhibitors, which have demonstrated desired efficacy and safety profile.

### SUMMARY

In one embodiment, the present invention provides novel compounds of formula (I),

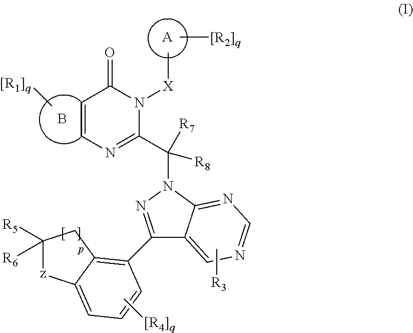

wherein,
X is a bond or

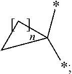

* denotes point of attachment to ring A and to the rest of molecule;
   when X is a bond, ring A is a mono or bicyclic heteroaryl containing at least one N and said N is point of attachment to X;
or when X is

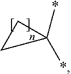

ring A is mono or bicyclic aryl or heteroaryl;
ring B is mono or bicyclic aryl or 6-membered heteroaryl;
   R₁ and R₂ are independently selected from hydrogen, halogen, NO₂, NR₁₁R₁₂, CF₃, CN, COOR₉, COR₉, OR₉, OCOR₉, O—(C₁-C₆)alkyl-OR₉, O—(C₁-C₆)alkyl-S(O)ₜR₉, O—(C₁-C₆)alkyl-NR₁₁R₁₂, O—(C₁-C₆)alkyl-COOR₉, O—(C₁-C₆)alkyl-COR₉, S(O)ₜR₉, (C₁-C₆)alkyl, (C₃-C₆)cycloalkyl, (C₁-C₆)alkyl-OR₉, (C₁-C₆)alkyl-S(O)ₜR₉, (C₁-C₆)alkyl-NR₁₁R₁₂, (C₁-C₆)alkylaryl, (C₁-C₆)alkylheteroaryl, (C₁-C₆)alkylheterocycloalkyl, (C₁-C₆)alkylcycloalkyl, (C₂-C₅)alkenyl-R₁₃, (C₂-C₅)alkynyl-R₁₃, heterocycloalkyl, aryl, boronate ester and heteroaryl; said (C₃-C₆)cycloalkyl, heterocycloalkyl, aryl and heteroaryl are optionally substituted by R₁₀;
   R₃ and R₄ are independently selected from hydrogen, OR₉, halogen, NR₁₁R₁₂, NO₂, CF₃, O—(C₁-C₆)alkyl-OR₉, O—(C₁-C₆)alkyl-S(O)ₜR₉, O—(C₁-C₆)alkyl-NR₁₁R₁₂, O—(C₁-C₆)alkyl-COOR₉, O—(C₁-C₆)alkyl-COR₉, S(O)ₜR₉, COR₉, COOR₉, (C₁-C₆)alkyl, (C₃-C₆)cycloalkyl, (C₂-C₅)alkenyl-R₁₃, (C₂-C₅)alkynyl-R₁₃ aryl, heteroaryl and heterocycloalkyl; the said (C₃-C₆)cycloalkyl, heterocycloalkyl, aryl and heteroaryl are optionally substituted by R₁₀;
   R₅, R₆, R₇ and R₈ are independently selected from hydrogen, halogen, NR₁₁R₁₂, CF₃, COOR₉, COR₉, and (C₁-C₆)alkyl, or
   R₅ and R₆ or R₇ and R₈ together may form 3 to 6 membered monocyclic cycloalkyl ring;
   R₉ is independently selected from hydrogen, NR₁₁R₁₂, CF₃, SO₃H, glucuronate, (C₁-C₆)alkyl, (C₁-C₆)alkyl-R₁₀, (C₃-C₆)cycloalkyl, heterocycloalkyl, aryl and heteroaryl; said (C₃-C₆)cycloalkyl, heterocycloalkyl, aryl and heteroaryl are optionally substituted by R₁₀;
   R₁₀ is independently selected from hydrogen, oxo, halogen, CF₃, S(O)ₜR₉, OR₉, NO₂, COR₉, COOR₉, NR₁₁R₁₂, N(R₉)COR₉, N(R₉)S(O)ₘR₉, OCOR₉, (C₁-C₆)alkyl, (C₃-C₆)cycloalkyl, (C₁-C₆)alkyl-OR₉, (C₁-C₆)alkyl-COOR₉, (C₁-C₆)alkyl-COR₉, (C₁-C₆)alkyl-S(O)ₜR₉, (C₁-C₆)alkyl-NHCOR₉, (C₁-C₆)alkyl-NHS(O)ₜR₉, (C₁-C₆)alkyl-NR₁₁R₁₂, heterocycloalkyl, aryl, heteroaryl, (C₂-C₅)alkenyl-R₁₃ and (C₂-C₅)alkynyl-R₁₃;
   R₁₁ and R₁₂ are independently selected from hydrogen, COR₉, N(R₉)₂, N(R₉)S(O)ₜR₉, N(R₉)COR₉, CF₃, S(O)ₜR₉, (C₁-C₆)alkyl, fluoro(C₁-C₆)alkyl, aryl, heteroaryl, heterocycloalkyl, (C₃-C₆)cycloalkyl, (C₁-C₆)alkyl(C₃-C₆)cycloalkyl, (C₁-C₆)alkylheterocycloalkyl, (C₁-C₆)alkylaryl, (C₁-C₆)alkylheteroaryl, (C₁-C₆)alkyl-OR₉, (C₁-C₆)alkyl-S(O)ₜR₉, (C₁-C₆)alkyl-COOR₉, (C₁-C₆)alkyl-COR₉, (C₁-C₆)alkyl-OCOOR₉, (C₁-C₆)alkyl-N(R₉)COR₉ and (C₁-C₆)alkyl-N(R₉)S(O)ₘR₉; or
   R₁₁ and R₁₂ together with N, may form a 3 to 8 member monocyclic or 8 to 12 membered bicyclic heterocycle ring, wherein the said mono and bicyclic ring may additionally contain 1, 2 and 3 ring heteroatoms selected from O, S(O)ₜ or N; said heterocycle is optionally substituted by R₁₀;
   R₁₃ is independently selected from hydrogen, (C₁-C₆)alkyl-OR₉, (C₁-C₆)alkyl-S(O)ₜR₉, (C₁-C₆)alkyl-COOR₉, (C₁-C₆)alkyl-COR₉, (C₁-C₆)alkyl-OCOOR₉, (C₁-C₆)alkyl-N(R₉)COR₉, (C₁-C₆)alkyl-N(R₉)S(O)ₘR₉, (C₁-C₆)alkyl-NR₁₁R₁₂, aryl, heteroaryl, (C₃-C₆)cycloalkyl, heterocycloalkyl, (C₁-C₆)alkyl(C₃-C₆)cycloalkyl, (C₁-C₆)alkylheterocycloalkyl, (C₁-C₆)alkylheteroaryl and (C₁-C₆)alkylaryl; said C₁-C₆)alkyl(C₃-C₆)cycloalkyl, (C₁-C₆)alkylheterocycloalkyl, (C₁-C₆)alkylheteroaryl, (C₁-C₆)alkylaryl, (C₃-C₆)cycloalkyl, heterocycloalkyl, aryl and heteroaryl are optionally substituted by R₁₀;
Z is CH₂ or O;
q is 1-3;
n is selected from 1-4;
p and m are independently 1 or 2;
and t is selected from 0-2;
   or pharmaceutically acceptable salts, metabolites, isomers, stereoisomers, atropisomers, conformers, tautomers, polymorphs, hydrates or solvates thereof.

In another embodiment, the present invention pertains to a compound as above, however only including pharmaceutically acceptable salts thereof.

In another embodiment, the present invention includes synthetic intermediates that are useful in preparing the compounds of formula (I) and process for preparing such intermediates.

Another embodiment of the present invention is a method for preparation of a compound of formula (I) or intermediates as herein described in Schemes 1 to 5.

Another embodiment of the present invention is a pharmaceutical composition comprising a compound of formula (I), optionally in admixture with a pharmaceutically acceptable adjuvant or carrier.

Another embodiment of the present invention is a method for treating allergic or non-allergic airway diseases by administering a therapeutically effective amount of a compound of formula (I) to a mammal, including human being, in need thereof.

Another embodiment of the present invention is a method for treating chronic obstructive pulmonary disease or asthma by administering a therapeutically effective amount of a compound of formula (I) to a mammal, including human being, in need thereof.

Another embodiment of the present invention is the use of a compound of formula (I) for the preparation of a medicament for treating allergic or non-allergic airway diseases.

Another embodiment of the present invention is the use of a compound of formula (I) for the preparation of a medicament for treating chronic obstructive pulmonary disease or asthma.

### BRIEF DESCRIPTION OF THE FIGURE

FIG. 1: Illustrates the effect of treatment with Compound No. 44 on OVA induced pulmonary inflammatory cell accumulation in BAL fluid. (Values represented are in mean±SEM, *p<0.05, ***p<0.001 vs saline; #p<0.05 versus vehicle)

### DETAILED DESCRIPTION

In one embodiment, the present invention provides novel compounds of formula (I),

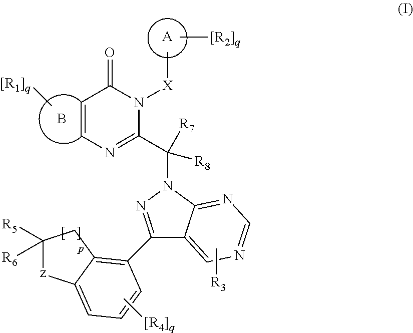

wherein R₁, R₂, R₃, R₄, R₅, R₆, R₇, R₈, A, B, X, q, z and p are as defined above, or pharmaceutically acceptable salts, metabolites, isomers, stereoisomers, atropisomers, conformers, tautomers, polymorphs, hydrates and solvates thereof.

In another embodiment, the present invention provides novel compounds of formula (Ia),

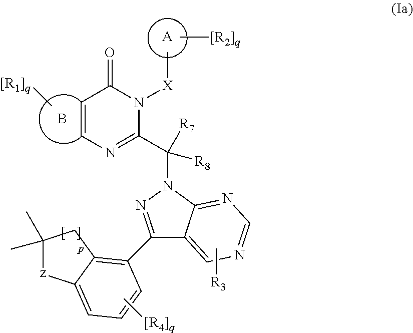

wherein R₁, R₂, R₃, R₄, R₇, R₈, A, B, X, z, q and p are as defined above or pharmaceutically acceptable salts, metabolites, isomers, stereoisomers, atropisomers, conformers, tautomers, polymorphs, hydrates and solvates thereof.

In a preferred embodiment, the present invention provides novel compounds of formula (I) or (Ia),
wherein
R₁, R₂, R₃, R₄, R₅, R₆, R₇, R₈, A, B, X, q are as defined above, z is O and p is 1;
   or pharmaceutically acceptable salts, metabolites, isomers, stereoisomers, atropisomers, conformers, tautomers, polymorphs, hydrates and solvates thereof.

In another preferred embodiment, the present invention provides novel compounds of formula (I),

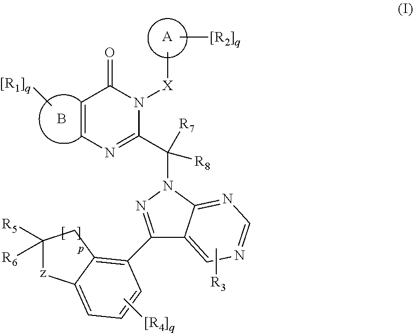

wherein,
X is a bond or

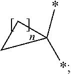

* denotes point of attachment to ring A and to the rest of molecule;
when X is a bond, ring A is a mono or bicyclic heteroaryl containing at least one N and said N is point of attachment to X;
or when X is

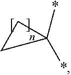

ring A is monocyclic aryl or heteroaryl;
ring B is monocyclic aryl;
   R₁ and R₂ are independently selected from hydrogen, halogen, CF₃, (C₁-C₆)alkyl, (C₃-C₆)cycloalkyl, (C₂-C₅)alkynyl-R₁₃, aryl and heteroaryl; said (C₃-C₆)cycloalkyl, aryl and heteroaryl are optionally substituted by R₁₀;
   R₃ and R₄ are independently selected from hydrogen, NR₁₁R₁₂ and OR₉;
   R₅, R₆, R₇ and R₈ are independently selected from hydrogen and (C₁-C₆)alkyl,
   R9 is independently selected from hydrogen, (C₁-C₆)alkyl and (C₃-C₆)cycloalkyl; said (C₃-C₆)cycloalkyl is optionally substituted by R₁₀;
   R₁₀ is independently selected from hydrogen, oxo, OR₉, and (C₁-C₆)alkyl;
   R₁₁ and R₁₂ are independently selected from hydrogen and (C₁-C₆)alkyl;
   R₁₃ is independently selected from hydrogen, (C₁-C₆)alkyl-OR₉, (C₁-C₆)alkyl-N(R₉)COR₉, (C₁-C₆)alkyl-NR₁₁R₁₂, heteroaryl, (C₃-C₆)cycloalkyl, (C₁-C₆)alkyl(C₃-C₆)cycloalkyl, (C₁-C₆)alkylheterocycloalkyl; said (C₃-C₆)cycloalkyl, (C₁-C₆)alkyl(C₃-C₆)cycloalkyl, (C₁-C₆)alkylheterocycloalkyl, and heteroaryl are optionally substituted by R₁₀;
Z is CH₂ or O;
n is 1;
q is 1 to 2;
and p is 1 or 2;
   or pharmaceutically acceptable salts, metabolites, isomers, stereoisomers, atropisomers, conformers, tautomers, polymorphs, hydrates and solvates thereof.

A family of specific compounds of particular interest within the scope of present invention consists of compound and pharmaceutically acceptable salts thereof as follows:

| Compd. No. | Chemical Name |
|---|---|
| 1 | 2-{[4-amino-3-(7-hydroxy-2,3-dihydro-1H-inden-4-yl)-1H-pyrazolo[3,4- |
| | d]pyrimidin-1-yl]methyl}-5-chloro-3-(1H-pyrrol-1-yl)quinazolin-4(3H)-one |
| 2 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-methyl-3-(1H-pyrrol-1- |
| | yl)quinazolin-4(3H)-one |
| 3 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-chloro-3-(1H-pyrrol-1- |
| | yl)quinazolin-4(3H)-one |
| 4 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-chloro-3-(2H-indazol-2- |
| | yl)quinazolin-4(3H)-one |
| 5 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-chloro-3-(1- |
| | phenylcyclopropyl)quinazolin-4(3H)-one |
| 6 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-chloro-3-(1H-pyrazol-1- |
| | yl)quinazolin-4(3H)-one |
| 7 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-methyl-3-(1H-pyrazol-1- |
| | yl)quinazolin-4(3H)-one |
| 8 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-methyl-3-(1- |
| | phenylcyclopropyl)quinazolin-4(3H)-one |
| 9 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-bromo-3-(1H-pyrrol-1- |
| | yl)quinazolin-4(3H)-one |
| 10 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(pyridin-3-yl)-3-(1H-pyrrol-1- |
| | yl)quinazolin-4(3H)-one |
| 11 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(3,5-dimethyl-1H-pyrazol-1- |
| | yl)-5-methylquinazolin-4(3H)-one |
| 12 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-chloro-3-(3,5-dimethyl-1H- |
| | pyrazol-1-yl)quinazolin-4(3H)-one |
| 13 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-bromo-3-(1- |
| | phenylcyclopropyl)quinazolin-4(3H)-one |
| 14 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(1H-pyrrol-1-yl)-5-(thiophen- |
| | 3-yl)quinazolin-4(3H)-one |
| 15 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(5-methylfuran-2-yl)-3-(1H- |
| | pyrrol-1-yl)quinazolin-4(3H)-one |
| 16 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(1-phenylcyclopropyl)-5- |
| | (pyridin-3-yl)quinazolin-4(3H)-one |
| 17 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(5-methylfuran-2-yl)-3-(1- |
| | phenylcyclopropyl)quinazolin-4(3H)-one |
| 18 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(pyridin-4-yl)-3-(1H-pyrrol-1- |
| | yl)quinazolin-4(3H)-one |
| 19 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(3-hydroxyprop-1-yn-1-yl)-3- |
| | (1H-pyrrol-1-yl)quinazolin-4(3H)-one |
| 20 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-ethynyl-3-(1H-pyrrol-1- |
| | yl)quinazolin-4(3H)-one |
| 21 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(1-phenylcyclopropyl)-5- |
| | (pyridin-4-yl)quinazolin-4(3H)-one |
| 22 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(1-methyl-1H-pyrazol-5-yl)-3- |
| | (1H-pyrrol-1-yl)quinazolin-4(3H)-one |
| 23 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(furan-3-yl)-3-(1H-pyrrol-1- |
| | yl)quinazolin-4(3H)-one |
| 24 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(3-hydroxyprop-1-yn-1-yl)-3- |
| | (1-phenylcyclopropyl)quinazolin-4(3H)-one |
| 25 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(1-phenylcyclopropyl)-5- |
| | (thiophen-3-yl)quinazolin-4(3H)-one |
| 26 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-fluoro-3-(1- |
| | phenylcyclopropyl)quinazolin-4(3H)-one |
| 27 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-6-methyl-3-(1H-pyrrol-1- |
| | yl)quinazolin-4(3H)-one |
| 28 | N-{3-[2-{[4-amino-3-(7-hydroxy-2,2-dimethyl-2,3-dihydro-1-benzofuran-4- |
| | yl)-1H-pyrazolo[3,4-d]pyrimidin-1-yl]methyl}-4-oxo-3-(1H-pyrrol-1-yl)- |
| | 3,4-dihydroquinazolin-5-yl]prop-2-yn-1-yl}acetamide |
| 29 | N-{3-[2-{[4-amino-3-(7-hydroxy-2,2-dimethyl-2,3-dihydro-1-benzofuran-4- |
| | yl)-1H-pyrazolo[3,4-d]pyrimidin-1-yl]methyl}-4-oxo-3-(1H-pyrrol-1-yl)- |
| | 3,4-dihydroquinazolin-5-yl]prop-2-yn-1-yl}cyclopropanecarboxamide |
| 30 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(1-methyl-1H-pyrazol-5-yl)-3- |
| | (1-phenylcyclopropyl)quinazolin-4(3H)-one |
| 31 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(3-hydroxyprop-1-yn-1-yl)-3- |
| | [1-(3-methylphenyl)cyclopropyl]quinazolin-4(3H)-one |
| 32 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-8-methyl-3-(1H-pyrrol-1- |
| | yl)quinazolin-4(3H)-one |
| 33 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-[1-(3- |
| | methylphenyl)cyclopropyl]-5-(1-methyl-1H-pyrazol-5-yl)quinazolin-4(3H)- |
| | one |
| 34 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-[3-(diethylamino)prop-1-yn-1- |
| | yl]-3-(1H-pyrrol-1-yl)quinazolin-4(3H)-one |
| 35 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-[3-(morpholin-4-yl)prop-1-yn- |
| | 1-yl]-3-(1H-pyrrol-1-yl)quinazolin-4(3H)-one |
| 36 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-cyclopropyl-3-(1H-pyrrol-1- |
| | yl)quinazolin-4(3H)-one |
| 37 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(3-hydroxyprop-1-yn-1-yl)-3- |
| | [1-(4-methylphenyl)cyclopropyl]quinazolin-4(3H)-one |
| 38 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-[3-(diethylamino)prop-1-yn-1- |
| | yl]-3-(1-phenylcyclopropyl)quinazolin-4(3H)-one |
| 39 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-methylquinazolin-4(3H)-one |
| 40 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-[1-(4- |
| | methylphenyl)cyclopropyl]-5-(1-methyl-1H-pyrazol-5-yl)quinazolin-4(3H)- |
| | one |
| 41 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-(1-methyl-1H-pyrazol-5-yl)quinazolin-4(3H)-one |
| 42 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-(3-hydroxyprop-1-yn-1-yl)quinazolin-4(3H)-one |
| 43 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(cyclopropylethynyl)-3-(2,5- |
| | dimethyl-1H-pyrrol-1-yl)quinazolin-4(3H)-one |
| 44 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(1-methyl-1H-pyrazol-4-yl)-3- |
| | (1H-pyrrol-1-yl)quinazolin-4(3H)-one |
| 45 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(1-ethyl-1H-pyrazol-4-yl)-3- |
| | (1H-pyrrol-1-yl)quinazolin-4(3H)-one |
| 46 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-6-methyl-3-(1- |
| | phenylcyclopropyl)quinazolin-4(3H)-one |
| 47 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-[3-(morpholin-4-yl)prop-1-yn- |
| | 1-yl]-3-(1-phenylcyclopropyl)quinazolin-4(3H)-one |
| 48 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(cyclopropylethynyl)-3-(1H- |
| | pyrrol-1-yl)quinazolin-4(3H)-one |
| 49 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(3-hydroxy-3-methylbut-1-yn- |
| | 1-yl)-3-(1H-pyrrol-1-yl)quinazolin-4(3H)-one |
| 50 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(3-hydroxy-3-methylbut-1-yn- |
| | 1-yl)-3-[1-(4-methylphenyl)cyclopropyl]quinazolin-4(3H)-one |
| 51 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(cyclopropylethynyl)-3-[1-(4- |
| | methylphenyl)cyclopropyl]quinazolin-4(3H)-one |
| 52 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-ethyl-3-(1H-pyrrol-1- |
| | yl)quinazolin-4(3H)-one |
| 53 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(3-hydroxy-3-methylbut-1-yn- |
| | 1-yl)-3-(1-phenylcyclopropyl)quinazolin-4(3H)-one |
| 54 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(cyclopropylethynyl)-3-(1- |
| | phenylcyclopropyl)quinazolin-4(3H)-one |
| 55 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-chloro-3-[1-(2- |
| | methylphenyl)cyclopropyl]quinazolin-4(3H)-one |
| 56 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-methyl-3-[1-(2- |
| | methylphenyl)cyclopropyl]quinazolin-4(3H)-one |
| 57 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-[3-(4-methylpiperazin-1-yl)prop-1-yn-1-yl]quinazolin-4(3H)-one |
| 58 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-[3-(3-oxopiperazin-1-yl)prop- |
| | 1-yn-1-yl]-3-(1H-pyrrol-1-yl)quinazolin-4(3H)-one |
| 59 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-[1-(2- |
| | methylphenyl)cyclopropyl]-5-(1-methyl-1H-pyrazol-5-yl)quinazolin-4(3H)- |
| | one |
| 60 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-methyl-3-[1-(thiophen-2- |
| | yl)cyclopropyl]quinazolin-4(3H)-one |
| 61 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-[3-(4-methylpiperazin-1- |
| | yl)prop-1-yn-1-yl]-3-(1H-pyrrol-1-yl)quinazolin-4(3H)-one |
| 62 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-(1-methyl-1H-pyrazol-4-yl)quinazolin-4(3H)-one |
| 63 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(1H-pyrrol-1-yl)-5- |
| | (trifluoromethyl)quinazolin-4(3H)-one |
| 64 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-[(1-methyl-1H-imidazol-5- |
| | yl)ethynyl]-3-(1H-pyrrol-1-yl)quinazolin-4(3H)-one |
| 65 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(cyclopropylethynyl)-3-[1-(2- |
| | methylphenyl)cyclopropyl]quinazolin-4(3H)-one |
| 66 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(1-methyl-1H-pyrazol-5-yl)-3- |
| | [1-(thiophen-3-yl)cyclopropyl]quinazolin-4(3H)-one |
| 67 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-[(1-methyl-1H-imidazol-5-yl)ethynyl]quinazolin-4(3H)-one |
| 68 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-[3-(3-oxopiperazin-1-yl)prop-1-yn-1-yl]quinazolin-4(3H)-one |
| 69 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(1-methyl-1H-pyrazol-4-yl)-3- |
| | [1-(thiophen-3-yl)cyclopropyl]quinazolin-4(3H)-one |
| 70 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-[1-(2- |
| | methylphenyl)cyclopropyl]-5-[3-(morpholin-4-yl)prop-1-yn-1-yl]quinazolin- |
| | 4(3H)-one |
| 71 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(1-methyl-1H-pyrrol-2-yl)-3- |
| | (1H-pyrrol-1-yl)quinazolin-4(3H)-one |
| 72 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-[3-(morpholin-4-yl)prop-1-yn- |
| | 1-yl]-3-[1-(thiophen-3-yl)cyclopropyl]quinazolin-4(3H)-one |
| 73 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(3-cyclopentylprop-1-yn-1-yl)- |
| | 3-(1H-pyrrol-1-yl)quinazolin-4(3H)-one |
| 74 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(3-hydroxyphenyl)-3-(1H- |
| | pyrrol-1-yl)quinazolin-4(3H)-one |
| 75 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(1-methyl-1H-pyrrol-2-yl)-3- |
| | [1-(thiophen-3-yl)cyclopropyl]quinazolin-4(3H)-one |
| 76 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(cyclopropylethynyl)-3-[1- |
| | (thiophen-3-yl)cyclopropyl]quinazolin-4(3H)-one |
| 77 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-(1-methyl-1H-pyrrol-2-yl)quinazolin-4(3H)-one |
| 78 | 2-{[4-amino-3-(7-methoxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-methylquinazolin-4(3H)-one |
| 79 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-[3-(4-methylpiperazin-1- |
| | yl)prop-1-yn-1-yl]-3-(1-phenylcyclopropyl)quinazolin-4(3H)-one |
| 80 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-[3-(morpholin-4-yl)prop-1-yn-1-yl]quinazolin-4(3H)-one |
| 81 | 2-{[4-amino-3-(7-hydroxy-2,3-dihydro-1H-inden-4-yl)-1H-pyrazolo[3,4- |
| | d]pyrimidin-1-yl]methyl}-5-methyl-3-[1-(2-methylphenyl)cyclopropyl] |
| | quinazolin-4(3H)-one |
| 82 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-[3-(3-oxopiperazin-1-yl)prop- |
| | 1-yn-1-yl]-3-(1-phenylcyclopropyl)quinazolin-4(3H)-one |
| 83 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(1-phenylcyclopropyl)-5-[3- |
| | (thiomorpholin-4-yl)prop-1-yn-1-yl]quinazolin-4(3H)-one |
| 84 | 2-{[4-amino-3-(7-methoxy-2,2-dimethyl-2,3-dihydro-1-benzofuran-4-yl)- |
| | 1H-pyrazolo[3,4-d]pyrimidin-1-yl]methyl}-5-methyl-3-(1H-pyrrol-1- |
| | yl)quinazolin-4(3H)-one |
| 85 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-(1-ethyl-1H-pyrazol-4-yl)quinazolin-4(3H)-one |
| 86 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-(1H-pyrazol-4-yl)quinazolin-4(3H)-one |
| 87 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-bromo-3-(2,5-dimethyl-1H- |
| | pyrrol-1-yl)quinazolin-4(3H)-one |
| 88 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1- |
| | yl)quinazolin-4(3H)-one |
| 89 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-(1-propyl-1H-pyrazol-4-yl)quinazolin-4(3H)-one |
| 90 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-(1-ethyl-1H-pyrazol-4-yl)-3- |
| | (1-phenylcyclopropyl)quinazolin-4(3H)-one |
| 91 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-3-(2,5-dimethyl-1H-pyrrol-1-yl)- |
| | 5-(thiophen-3-yl)quinazolin-4(3H)-one |
| 92 | 2-{(1S)-1-[4-amino-3-(7-hydroxy-2,2-dimethyl-2,3-dihydro-1-benzofuran-4- |
| | yl)-1H-pyrazolo[3,4-d]pyrimidin-1-yl]ethyl}-5-methyl-3-(1H-pyrrol-1- |
| | yl)quinazolin-4(3H)-one |
| 93 | 2-{[4-amino-3-(7-hydroxy-2,3-dihydro-1H-inden-4-yl)-1H-pyrazolo[3,4- |
| | d]pyrimidin-1-yl]methyl}-3-(2,5-dimethyl-1H-pyrrol-1-yl)-5-(1-methyl-1H- |
| | pyrazol-4-yl)quinazolin-4(3H)-one |
| 94 | 2-{[4-amino-3-(7-hydroxy-2,3-dihydro-1H-inden-4-yl)-1H-pyrazolo[3,4- |
| | d]pyrimidin-1-yl]methyl}-3-(2,5-dimethyl-1H-pyrrol-1-yl)-5-(1-ethyl-1H- |
| | pyrazol-4-yl)quinazolin-4(3H)-one |
| 95 | 2-{(1S)-1-[4-amino-3-(7-hydroxy-2,2-dimethyl-2,3-dihydro-1-benzofuran-4- |
| | yl)-1H-pyrazolo[3,4-d]pyrimidin-1-yl]ethyl}-3-(2,5-dimethyl-1H-pyrrol-1- |
| | yl)-5-methylquinazolin-4(3H)-one |
| 96 | 2-{(1RS)-1-[4-amino-3-(7-hydroxy-2,2-dimethyl-2,3-dihydro-1-benzofuran- |
| | 4-yl)-1H-pyrazolo[3,4-d]pyrimidin-1-yl]ethyl}-5-methyl-3-(1H-pyrrol-1- |
| | yl)quinazolin-4(3H)-one |
| 97 | 2-{(1S)-1-[4-amino-3-(7-hydroxy-2,2-dimethyl-2,3-dihydro-1-benzofuran-4- |
| | yl)-1H-pyrazolo[3,4-d]pyrimidin-1-yl]ethyl}-5-methyl-3-[1-(3- |
| | methylphenyl)cyclopropyl]quinazolin-4(3H)-one |
| 98 | 2-{(1S)-1-[4-amino-3-(7-hydroxy-2,3-dihydro-1H-inden-4-yl)-1H- |
| | pyrazolo[3,4-d]pyrimidin-1-yl]ethyl}-5-methyl-3-(1H-pyrrol-1- |
| | yl)quinazolin-4(3H)-one |
| 99 | 2-{(1S)-1-[4-amino-3-(7-hydroxy-2,2-dimethyl-2,3-dihydro-1-benzofuran-4- |
| | yl)-1H-pyrazolo[3,4-d]pyrimidin-1-yl]ethyl}-5-(1-ethyl-1H-pyrazol-4-yl)-3- |
| | (1H-pyrrol-1-yl)quinazolin-4(3H)-one |
| 100 | 2-{[4-amino-3-(7-hydroxy-2,2-dimethyl-2,3-dihydro-1-benzofuran-4-yl)- |
| | 1H-pyrazolo[3,4-d]pyrimidin-1-yl]methyl}-5-methyl-3-(1H-pyrrol-1- |
| | yl)quinazolin-4(3H)-one methanesulfonate |
| 101 | 4-(4-amino-1-{[5-methyl-4-oxo-3-(1H-pyrrol-1-yl)-3,4-dihydroquinazolin- |
| | 2-yl]methyl}-1H-pyrazolo[3,4-d]pyrimidin-3-yl)-2,2-dimethyl-2,3-dihydro- |
| | 1-benzofuran-7-yl L-glucopyranosiduronic acid |
| 102 | 4-(4-amino-1-{[5-methyl-4-oxo-3-(1H-pyrrol-1-yl)-3,4-dihydroquinazolin- |
| | 2-yl]methyl}-1H-pyrazolo[3,4-d]pyrimidin-3-yl)-2,2-dimethyl-2,3-dihydro- |
| | 1-benzofuran-7-yl hydrogen sulfate |

### Definitions

The following definitions apply to the terms as used throughout this specification, unless otherwise limited in specific instances:

The term “compound” employed herein refers to any compound encompassed by the generic formula disclosed herein. The compounds described herein may contain one or more double bonds and therefore, may exist as isomers, stereoisomers, such as geometric isomers, E and Z isomers, and may possess asymmetric carbon atoms (optical centres) and therefore may exist as enantiomers, diastereoisomers. Accordingly, the chemical structures described herein encompasses all possible stereoisomers of the illustrated compounds including the stereoisomerically pure form (e.g., geometrically pure) and stereoisomeric mixtures (racemates). The compound described herein, may exist as conformational isomers such as chair or boat form. The compound described herein may also exist as atropisomers. The compounds may also exist in several tautomeric forms including the enol form, the keto form and mixtures thereof. Accordingly, the chemical structures described herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to ²H, ³H, ¹³C, ¹⁴C, ¹⁵N, ¹⁸O, ¹⁷O, etc. Compounds may exist in unsolvated forms as well as solvated forms, including hydrated forms. In general, compounds may be hydrated or solvated. Certain compounds may exist in multiple crystalline or amorphous forms. In general, all physical forms are equivalent for the uses contemplated herein and are intended to be within the scope of the present invention.

The use of the terms “a” & “an” & “the” and similar referents in the context of describing the invention (especially in the context of the following claims) are to be construed to cover both the singular and the plural, unless otherwise indicated herein or clearly contradicted by context.

The nomenclature of the compounds of the present invention as indicated herein is according to ACD/Lab's ChemDraw with “logD Suite” (Version 12.0) “Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, carbonic acid, phosphoric acid, and the like; or formed with organic acids such as acetic acid, propionic acid, isobutyric acid, hexanoic acid, cyclopentanepropionic acid, oxalic acid, glycolic acid, pyruvic acid, lactic acid, malonic acid, succinic acid, suberic acid, malic acid, maleic acid, fumaric acid, tartaric acid, citric acid, benzoic acid, 3-(4-hydroxybenzoyl) benzoic acid, phthalic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glucuronic acid, galactunoric acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. Also included are salts of amino acids such as arginate and the like (see, for example, Berge, S. M., et al., “Pharmaceutical Salts”, Journal of Pharmaceutical Science, 1977, 66, 1-19).

As used herein, the term “polymorph” pertains to compounds having the same chemical formula, the same salt type and having the same form of hydrate/solvate but having different crystallographic properties.

As used herein, the term “hydrate” pertains to a compound having a number of water molecules bonded to the compound.

As used herein, the term “solvate” pertains to a compound having a number of solvent molecules bonded to the compound.

As used herein, the term “metabolites” pertains to the compounds formed in-vivo upon administration of the drug. Some examples of such metabolites according to present invention are compounds 101 and 102.

The present invention also encompasses compounds which are in a prodrug form. Prodrugs of the compounds described herein are those compounds that readily undergo chemical changes under physiological conditions (in vivo) to provide the compounds of the present invention. Additionally, prodrugs can be converted to the compounds of the present invention by chemical or biochemical methods in an ex vivo environment, for example, transdermal patch reservoir with a suitable enzyme or chemical. Prodrugs are, in some situation, easier to administer than the parent drug. They may, for instance, be bioavailable by oral administration whereas the parent drug is not. The prodrug may also have improved solubility in pharmacological composition over the parent drug. Esters, peptidyl derivatives and the like, of the compounds are the examples of prodrugs of the present invention. In vivo hydrolysable (or cleavable) ester of a compound of the present invention that contains a carboxy group is, for example, a pharmaceutically acceptable ester which is hydrolysed in the human or animal body to produce the parent acid.

The term “substituted”, as used herein, includes mono- and poly-substitution by a named substituent to the extent such single and multiple substitution (including multiple substitution at the same site) is chemically allowed and which means that any one or more hydrogen on the designated atom is replaced with a selection from the indicated group, provided that the designated atom's normal valence is not exceeded, and that the substitution results in a stable compound, for example, when a substituent is keto, then the two hydrogens on the atom are replaced. All substituents (R₁, R₂ . . . ) and their further substituents described herein may be attached to the main structure at any heteroatom or carbon atom which results in formation of stable compound.

As used herein, a “halogen” substituent is a monovalent halogen radical chosen from chloro, bromo, iodo and fluoro.

The term “(C₁-C₆)alkyl” used either alone or in attachment with another group refers to aliphatic hydrocarbon radical having the 1 to 6 carbon atoms and that is unsubstituted or substituted. Said “(C₁-C₆)alkyl” may be straight (for example, methyl, ethyl, n-propyl, n-butyl, n-pentyl, n-hexyl) or branched chain (for example, isopropyl, isobutyl, sec-butyl, tert-butyl) and it may contain one or two double or triple bonds to form corresponding alkenes or alkynes. The said (C₁-C₆)alkyl may also contain (C₃-C₆)cycloalkyl ring in a spiro manner.

The term “(C₃-C₆) cycloalkyl” used either alone or in attachment with another group refers to a cyclic ring system having the 3 to 6 carbon atoms and that is unsubstituted or substituted. The said “(C₃-C₆) cycloalkyl” means a cyclic ring system containing only carbon atom in the ring system backbone such as cyclopropyl, cyclobutyl, cyclopentyl, cyclohexyl.

Cycloalkyl may include bicyclic fused rings. Cycloalkyl may have any degree of saturation provided that at least one ring in the ring system is not aromatic.

The term “aryl” used either alone or in attachment with another group refers to an aromatic group for example, which is a 6 to 10 membered monocyclic or bicyclic carbon-containing ring system. The aryl groups include, but are not limited to, phenyl, naphthyl, biphenyl, tetrahydronaphthyl and indane. Preferably, aryl is phenyl.

The term “heteroaryl” used either alone or in attachment with another group refers to an aromatic group for example, which is a 5 to 14 membered monocyclic or bicyclic ring system, which has at least one heteroatom. The term “heteroatom” as used herein includes O, N, S. In bicyclic ring system, ring can be fused through a bridge heteroatom. The heteroaryl groups include, but are not limited to pyrrolyl, furanyl (furyl), thiophenyl (thienyl), pyrazolyl, imidazolyl, oxazolyl, isoxazolyl, thiazolyl, triazolyl, oxadiazolyl, thiadiazolyl, tetrazolyl, pyridinyl (pyridyl), pyridazinyl, pyrimdinyl, pyrazinyl, triazinyl, indolyl, benzofuranyl, benzothiophenyl (benzothienyl), indazolyl, benzimidazol, benzoxazolyl, benzisoxazolyl, benzothiazolyl, quinolinyl, isoquinolinyl, cinnolinyl, quinazolinyl, quinoxalinyl, phthalazinyl or naphthyridinyl.

The term “heterocycloalkyl” or “heterocycle” used either alone or in attachment with another group refers to a fully or partially saturated cyclic group, for example, which is a 3 to 14 membered monocyclic or bicyclic ring system, which has at least one heteroatom. The term “heteroatom” as used herein includes O, N, S. In bicyclic heterocycloalkyl system, at least one ring is not aromatic and the rings can also be attached to each other in a spiro manner. The heterocycloalkyl or heterocycle groups include, but are not limited, oxiranyl, aziridinyl, oxetanyl, azetidinyl, pyrrolidinyl, dihydropyrrolyl, tetrahydrofuranyl, dihydrofuranyl, tetrahydrothiophenyl, dihydrothiophenyl, pyrazolidinyl, imidazolidinyl, oxazolidinyl, isoxazoiidinyl, thiazoiidinyl, triazolidinyl, oxadiazolidinyl, piperidinyl, tetrahydropyridinyl, dihydropyridinyl, piperazinyl, tetrahydropyranyl, dioxanyl, morpholinyl, triazinanyl, azepanyl, diazepanyl, diazepinyl, oxepanyl, dioxepanyl, oxazepanyl, oxazepinyl, indolinyl, benzomorpholinyl, tetrahydroquinolyl tetrahydrisoquinolyl or thiomorpholinyl.

As used herein, “room temperature” refers to a temperature between 20° C. and 30° C.

As used herein, the term “mammal” means a human or an animal such as monkeys, primates, dogs, cats, horses, cows, etc.

The terms “treating” or “treatment” of any disease or disorder as used herein to mean administering a compound to a mammal, including human being, in need thereof. The compound may be administered thereby providing a prophylactic effect in terms of completely or partially preventing or delaying the onset of a disease or disorder or sign or symptom thereof; and/or the compound may be administered thereby providing a partial or complete cure for a disease or disorder and/or adverse effect attributable to the disorder.

The phrase “a therapeutically effective amount” means the amount of a compound that, when administered to a patient for treating a disease, is sufficient to effect such treatment for the disease. The “therapeutically effective amount” will vary depending on the compound, mode of administration, the disease and its severity and the age, weight, etc., of the patient to be treated.

Throughout this specification and the appended claims, it is to be understood that the words “comprise” and “include” and variations such as “comprises”, “comprising”, “includes”, “including” are to be interpreted inclusively, unless the context requires otherwise. That is, the use of these words may imply the inclusion of an element or elements not specifically recited.

In another embodiment, present invention provides the process for preparing the compounds of formula (I).

The following reaction schemes are given to disclose the synthesis of the compounds according to the present invention.

Accordingly, the compounds of formula (I) or (Ia) of the present invention may be prepared as described in the schemes below.

Illustrative embodiments of compounds of formula (I) include compounds of formula I-A, formula I-B, formula I-C, formula I-D, formula I-E, formula I-F and I-G, in which the substituents are as defined in connection with general formula (I) or (Ia) and schemes 1-2.

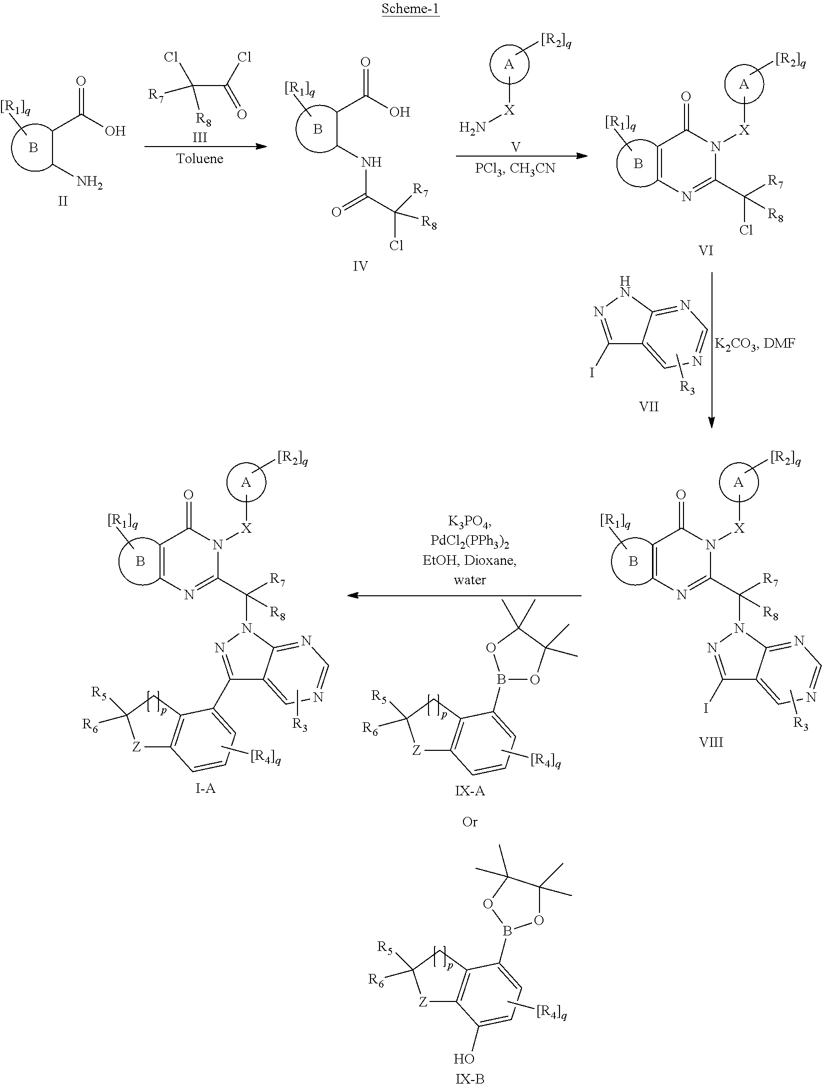

Synthesis of compound of formula I-A, where R₁ is hydrogen, halo, (C₁-C₆) alkyl etc, is shown in scheme 1. Compound of formula I-A can be prepared by the reaction of iodo derivative of formula VIII and Boronate ester of formula IX-A or IX-B in the presence of suitable catalyst such as dichlorobis(triphenylphosphine)palladium(II) (PdCl₂(PPh₃)₂)in suitable solvent such as ethanol, dioxane, toluene, DMF, water or mixture thereof and suitable base such as tripotassium phosphate at room temperature to reflux temperature. Compound of formula VIII can be prepared by the reaction of compound of formula VI with compound of formula VII in presence of suitable base such as potassium carbonate in suitable solvent such as DMF at room temperature. Compound of formula VI can be prepared by the reaction of an appropriate amino compound of formula V with acid compound of formula IV, in the presence of PCl₃ and suitable solvent such as acetonitrile, THF, DMF, dioxane or mixture thereof at room temperature to reflux temperature. Compound of formula IV can be prepared by the N-acetylation of compound of formula II with suitable halo compound of formula III, in suitable solvent such as toluene at room temperature to reflux temperature. Compound of formula II, with various R₁ substitutions (for example 2-amino-6-methylbenzoic acid, 2-amino-6-bromobenzoic acid and 2-aminonicotinic acid) are either commercially available or synthesized using conventional methods known to one of skill in the art. Similarly, various compounds of formula III, V or VII are either commercially available or can be synthesized using conventional methods known to one of skill in the art. Some of compounds of formula V such as 1H-indazol-1-amine and 2H-indazol-2-amine can be synthesized from appropriate starting material using similar procedure as described in J. Med. Chem. 2008, 51, 3599-3608.

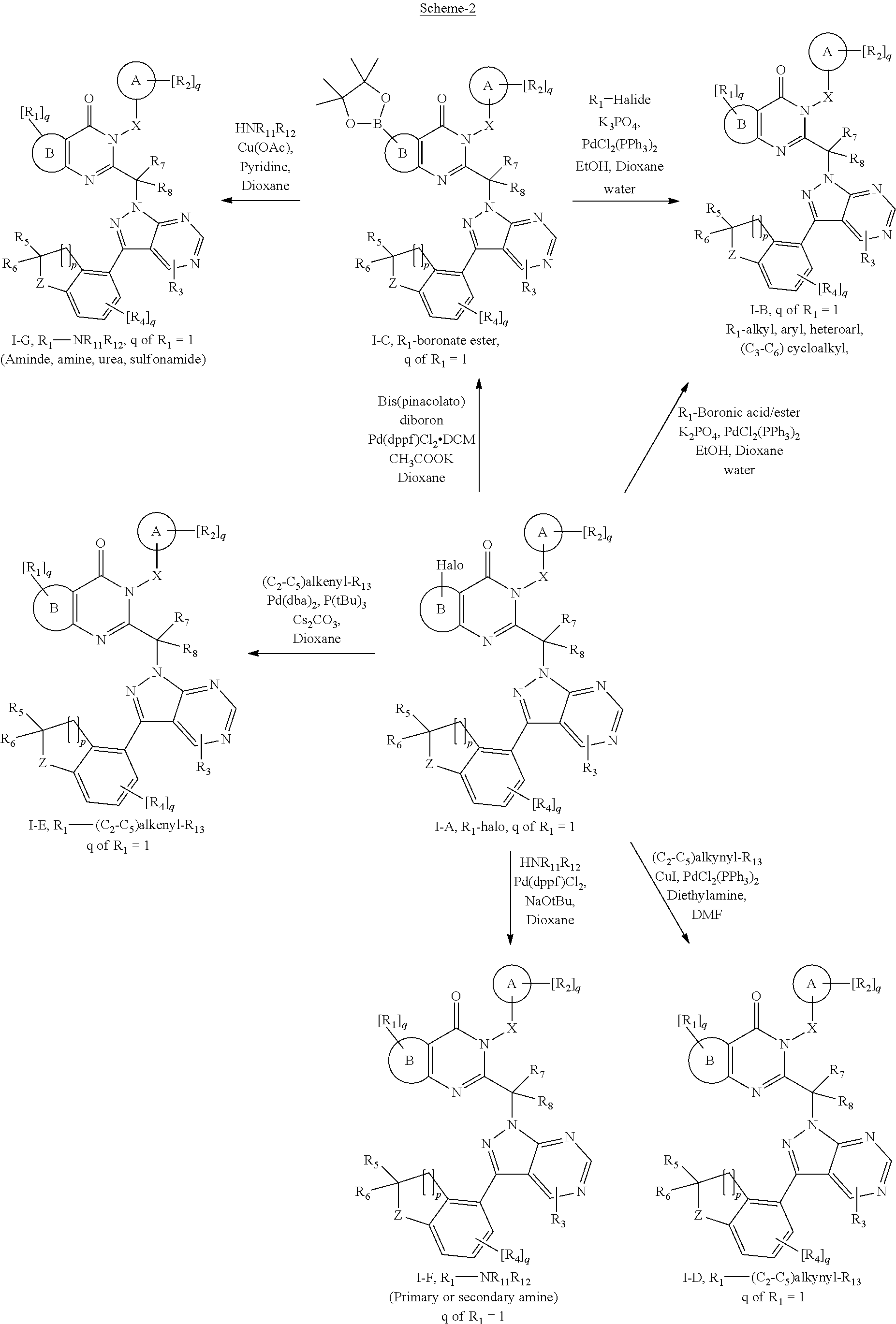

Synthesis of various compounds of formula I-B, I-C, I-D, I-E, I-F and I-G wherein R₁ is alkyl, aryl, heteroaryl, boronate ester, (C₃-C₆)cycloalkyl, (C₂-C₅)alkynyl-R₁₃, etc. is shown in scheme 2. Compounds of formula I-B, where R₁ is aryl, heteroaryl or (C₃-C₆)cycloalkyl, is prepared by the reaction between compound of formula I-A, where R₁ is halo, with appropriate R₁-boronic acid or ester in the presence of suitable catalyst such as bis(dibenzylideneacetone)palladium(0), tetrakis(triphenylphosphine)palladium(0) or dichlorobis(triphenylphosphine)palladium(II) in suitable solvent such as ethanol, dioxane, toluene,dimethyl formamide or mixture thereof along with water and suitable base such as tripotassium phosphate or sodium carbonate at room temperature to reflux temperature. Similarly compound of formula I-D, wherein R₁ is (C₂-C₅)alkynyl-R₁₃ can be prepared by the reaction between compound of formula I-A, where R₁ is halo, with appropriately substituted (C₂-C₅)alkynyl-R₁₃ derivatives in the presence of suitable catalyst such as bis(dibenzylideneacetone)palladium(0), tetrakis(triphenylphosphine)palladium(0) or dichlorobis(triphenylphosphine)palladium(II) in suitable solvent such as DMF along with copper iodide and suitable base such as diethylamine at room temperature to reflux temperature. In alternate pathway, compound of the formula I-B wherein R₁ is alkyl, aryl, heteroaryl and (C₃-C₆)cycloalkyl can be prepared by the reaction between compound of formula I-C, where R₁ is boronate ester, and appropriate R₁-halide in the presence of suitable catalyst such as Bis(dibenzylideneacetone)palladium(0), Tetrakis(triphenyl phosphine)palladium(0) or dichlorobis(triphenylphosphine)palladium(II) in suitable solvent such as ethanol, dioxane, toluene, dimethyl formamide or mixture thereof along with water and suitable base such as tripotassium phosphate or sodium carbonate at room to reflux temperature. Compound of formula I-C can be prepared by the reaction between I-A, where R₁ is halo, with bis(pinacolato)diboron in presence of catalyst such as 1,1′-bis(diphenylphosphino)ferrocene]dichloro palladium complex with dichloromethane (Pd(dppf)Cl₂.DCM) and suitable base such as potassium acetate in suitable solvent such as dioxane, DMSO, DMF or mixture thereof at room temperature to reflux temperature.

In similar way, compound of formula I-A, where R₁ is halo, can be treated with (C₂-C₅)alkenyl-R₁₃ using reaction conditions of Heck reaction, for instance in presence of appropriate palladium catalyst with suitable base such as sodium carbonate or potassium carbonate in solvent such as DMF, dioxane or mixture thereof to provide compounds of formula I-E, where R₁ is (C₂-C₅)alkenyl-R₁₃. Also compound of formula I-A, where R₁ is halo, can be treated with primary or secondary amine (HNR₁₁R₁₂) under the reaction condition of Buchwald-Hartwig cross coupling reaction, for instance in presence of appropriate palladium catalyst and base such as sodium tert-butoxide, K₃PO₄, K₂CO₃or potassium tert-butoxide in solvent such as dioxane, toluene, butanol or mixture thereof at room temperature to reflux temperature to yield compounds of formula I-F, where R₁ is NR₁₁R₁₂. Also compound of formula I-C, where R₁ is boronate ester, can be treated with appropriate amide or amine using reaction conditions of Chan-Lam coupling, for instance in presence of copper catalyst such as copper acetate or nickel based catalyst in presence of base such as pyridine or DMAP in solvent such as methylene dichloride, acetonitrile, toluene, methanol or mixture thereof at room temperature to reflux temperature to furnish compounds of formula I-G, where R₁ is NR₁₁R₁₂, and OR₉.

Intermediates used in preparation of compound of formula (I) or (Ia) can be prepared by Schemes 3 to 5. Illustrative embodiments of intermediate compounds of formula IX include compounds of formula IX-A and IX-B, in which substituents are defined in connection with General formula (I) or (Ia).

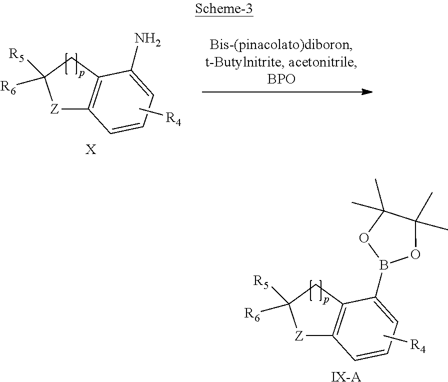

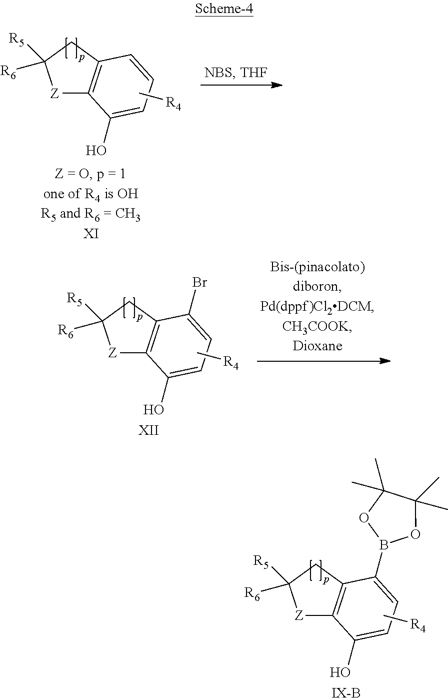

Synthesis of various compound of formula IX-A and IX-B is shown in scheme 3 and 4, respectively. Compound of formula IX-A can be prepared by the reaction of compound of formula X with bis(pinacolato)diboron in presence of tert-butyl nitrite and catalytic amount of benzoyl peroxide in solvent such as acetonitrile at 0° C. to room temperature. Compound of the formula IX-B can be prepared by the reaction of the compound of formula XII with bis(pinacolato)diboron using catalyst such as 1,1′-bis(diphenylphosphino)ferrocene]dichloro palladium complex with dichloromethane (Pd(dppf)Cl2.DCM) in presence of suitable base such as potassium acetate in solvent such as dioxane at room temperature to reflux temperature. Compound of formula XII can be prepared by the reaction of the compound of formula XI with N-bromosuccinimide (NBS) in solvent such as tetrahydrofuran at 0° C. to room temperature. Compound of formula X and XI are either commercially available or can be synthesized using conventional methods known to one of skill in the art. Some of compounds of formula X such as 7-amino-2,3-dihydro-1H-inden-4-ol and 4-amino-2,2-dimethyl-2,3-dihydro-1-benzofuran-7-ol can be synthesized from appropriate starting material using similar procedure as described in as described in U.S. Pat. No. 6,203,580.

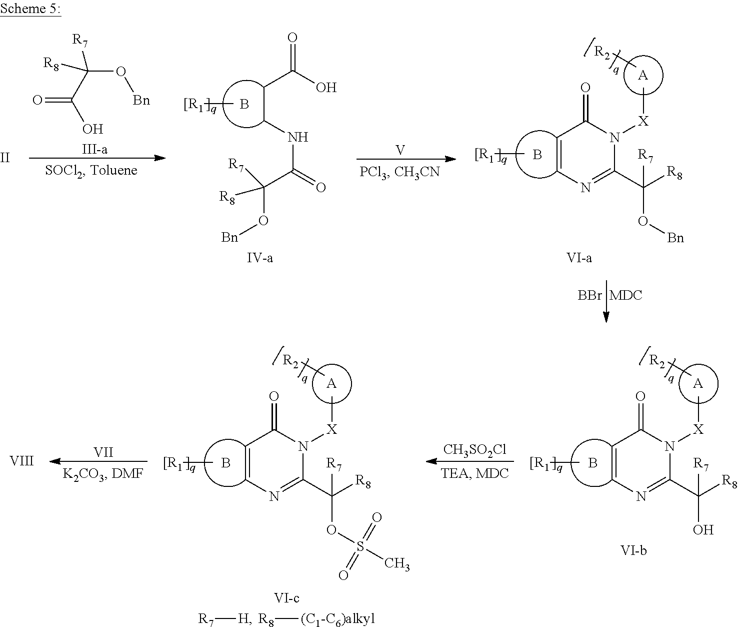

Synthesis of some of the intermediate compounds of formula VIII, wherein R7 and R8 are independently hydrogen and (C₁-C₆)alkyl, is shown in scheme 5. Compound of formula VIII can be prepared by the reaction of compound of formula VI-c with compound of formula VII in presence of base such as potassium carbonate in suitable solvent such as DMF at room temperature. Compound of formula VI-c can be prepared by the reaction of VI-b with methane sulfonyl chloride in presence of TEA in suitable solvent such as MDC, THF or dioxane at 0 to 10° C. Compound of formula VI-b can be prepared by the reaction of VI-a with BBr₃ in suitable solvent such as MDC at 0° C. Compound of formula VI-a can be obtained by the reaction of IV-a with V in presence of PCl₃ and suitable solvent such as acetonitrile or toluene at reflux temperature. Compound of formula IV-a can be prepared by reaction of II with III-a in presence of thionyl chloride and suitable solvent such as toluene or DMF at room to reflux temperature.

Schemes 1-5 given herein above provide general method of preparation of compounds and intermediates according to present invention. One of ordinary skill will recognize to appropriately substitute various groups such as R₁, R₂, R₃, R₄, R₅, R₆, R7 and R₈ in appropriately modified starting material to prepare desired compounds according to formula (I) or (Ia). Alternative to the given schemes, one of ordinary skill will readily synthesize the compounds according to the present invention using conventional synthetic organic techniques from suitable starting material which are either commercially available or may be readily prepared.

It would be obvious to one skilled in the art that variations in reaction time, temperature, solvents and/or reagents could increase the yields.

The compounds of the present invention may have chiral centers and occur as racemates, racemic mixtures and as individual diastereomers or enantiomers with all isomeric forms being included in the present invention. Therefore, where a compound is chiral, the separate enantiomers, substantially free of the other, are included within the scope of the invention; further included are all mixtures of the two enantiomers.

The novel compounds of the present invention were prepared according to the procedure of the schemes as described herein above, using appropriate materials and are further exemplified by the following specific examples. The examples are not to be considered or construed as limiting the scope of the invention set forth.

In present specification some general terms are used with their known intended meaning which are defined herein below:

| | |
|---|---|
| APCI | Atmospheric pressure chemical ionization |
| Bn | Benzyl |
| BPO | Benzoyl peroxide |
| CH₃CN or ACN | Acetonitrile |
| CH₃COOK | Potassium acetate |
| DBU | 1,8-diazabicyclo[5.4.0]undec-7-ene |
| DIEA | N,N-diisopropylethylamine |
| DMAP | 4-dimethylaminopyridine |
| DMF | Dimethyl formamide |
| EtOH | Ethanol |
| EtOAc | Ethyl acetate |
| ESMS | Electrospray Mass Spectrometry |
| ESI | Electro spray ionization |
| K₂CO₃ | Potassium carbonate |
| K₃PO₄ | Tripotassium phosphate |
| MDC or DCM | Methylene dichloride |
| ML | Mother liquor |
| PdCl₂(PPh₃)₂ | Bis(triphenylphosphine)palladium(II) dichloride |
| NBS | N-bromosuccninimide |
| NMR | Nuclear magnetic resonance |
| Pd(dppf)Cl₂•DCM | [1,1′-Bis(diphenylphosphino)ferrocene]- |
| | dichloropalladium(II), complex with |
| | dichloromethane |
| PCl₃ | Phosphorus trichloride |
| SOCl₂ | Thionyl Chloride |
| RT | Room temperature |
| RM | Reaction mixture |
| THF | Tetrahydrofuran |
| TEA | Triethylamine |
| TS | Tobacco smoke |
| COPD | Chronic obstructive pulmonary disease |
| BALF | Bronchoalveolar lavage fluid |
| Mg | Milligram |
| PBS | Phosphate buffer saline |
| NA | Not applicable |
| SEM | Standard error mean |

Mass of compounds prepared according to present invention is measured using Single quadrupole mass spectrometer (Water ZQ 2000 instrument) using APCI ionization technique (Electro spray chemical ionization Probe) or Finnigan LXQ, thermo instrument Technique using either ESI or APCI.

### EXAMPLES

### Example 1

### 2,2-dimethyl-4-(4,4,5,5-tetramethyl-1,3,2-dioxaborolan-2-yl)-2,3-dihydro-1-benzo furan-7-ol (Intermediate 1)

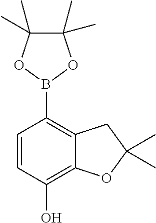

To a stirred solution of 4-amino-2,2-dimethyl-2,3-dihydro-1-benzofuran-7-ol (30 gm, 167 mmol) and bis(pinacolato)diboron (51 gm, 201 mmol) in acetonitrile (300 ml), catalytic amount of benzoyl peroxide was added at room temperature. The reaction mixture was cooled to 0° C. and tertiary butyl nitrite (30 ml, 251 mmol) was added drop wise to the reaction mixture. The reaction mixture was stirred at room temperature for 5 to 6 h. The reaction mixture was poured into water and extracted by ethyl acetate. Ethyl acetate layer was separated, dried over sodium sulfate and concentrated under vacuum to get crude solid. The crude product was purified further by silica gel column chromatography (Hexane/Ethyl acetate=90:10) to give 26.0 gm of title compound as white solid.

1H-NMR (400 MHz, DMSO-d6): δ 9.44 (1H, s), 6.95 (1H, d), 6.58 (1H, d), 3.06 (2H, s), 1.40 (6H, s), 1.25 (12H, s).

ESMS: 288.90 (M−2)

### Example 2

### 7-(4,4,5,5-tetramethyl-1,3,2-dioxaborolan-2-yl)-2,3-dihydro-1H-inden-4-ol (Intermediate 2)

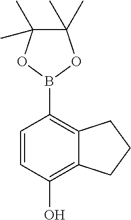 [SMILES: CC1(C)OB(c2ccc(O)c3c2CCC3)OC1(C)C]

Using the similar procedure as described in Example 1 for 2,2-dimethyl-4-(4,4,5,5-tetramethyl-1,3,2-dioxaborolan-2-yl)-2,3-dihydro-1-benzofuran-7-ol, title compound was synthesized from 7-amino-2,3-dihydro-1H-inden-4-ol and bis(pinacolato)diboron.

ESMS: 258.99 (M−2)

### Example 3

### 2-[(chloroacetyl)amino]-6-methylbenzoic acid (Intermediate 3)

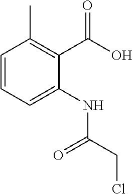

To a stirred solution of 2-amino-6-methylbenzoic acid (25 gm, 165 mmol) in toluene, (375 ml) chloroacetyl chloride (26 ml, 326 mmol) was added drop wise at room temperature. The reaction mixture was heated at 100° C. for 6 h. The reaction mixture was cooled to room temperature and poured into ice cooled water; the solid thus precipitated was filtered and dried under vacuum to get 30.0 gm of title compound as brown solid.

1H-NMR (400 MHz, DMSO-d6): δ 13.53 (1H, bs), 10.15 (1H, s), 7.63 (1H, d), 7.35 (1H, t), 7.11 (1H, d), 4.33 (2H, s), 2.37 (3H, s).

ESMS: 226.02 (M−1)

### Example 4

### 2-bromo-6-[(chloroacetyl)amino]benzoic acid (Intermediate 4)

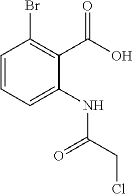 [SMILES: O=C(CCl)Nc1cccc(Br)c1C(=O)O]

Using the similar procedure as described in Example 3 for 2-[(chloroacetyl)amino]-6-methylbenzoic acid, the title compound was synthesized from 2-amino-6-bromobenzoic acid and chloro acetyl chloride.

1H-NMR (400 MHz, DMSO-d6): δ 13.80 (1H, bs), 9.99 (1H, s), 7.60 (1H, d), 7.54 (1H, d), 7.37 (1H, t), 4.31 (2H, s).

ESMS: 291.95 (M−1)

### Example 5

### 2-(chloromethyl)-5-methyl-3-(1H-pyrrol-1-yl)quinazolin-4(3H)-one (Intermediate 5)

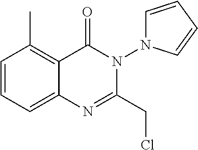 [SMILES: Cc1cccc2nc(CCl)n(-n3cccc3)c(=O)c12]

To a stirred solution of 2-[(chloroacetyl)amino]-6-methylbenzoic acid (Intermediate 3) (28.9 gm, 127 mmol) in acetonitrile (240 ml), phosphorus trichloride (22 ml, 254 mmol) was added drop wise at room temperature. 1H-pyrrol-1-amine (15.6 gm, 190 mmol) in acetonitrile (50 ml) added drop wise to the reaction mixture at room temperature. The reaction mixture was heated at 80° C. for 2 h. The reaction mixture was cooled to room temperature and poured into ice cooled water and stirred at room temperature for 1 h. The solid thus precipitated was filtered, washed by water and dried under vacuum to get 29 gm of title compound as light brown solid.

1H-NMR (400 MHz, DMSO-d6): δ 7.68-7.71 (1H, m), 7.60 (1H, d), 7.41 (1H, d), 7.05 (2H, t), 6.25 (2H, t), 4.32 (2H, s), 2.74 (3H, s).

ESMS: 274.04 (M+1)

### Example 6

### 5-bromo-2-(chloromethyl)-3-(1H-pyrrol-1-yl)quinazolin-4(3H)-one (Intermediate 6)

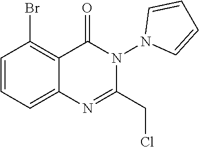 [SMILES: O=c1c2c(Br)cccc2nc(CCl)n1-n1cccc1]

Using the similar procedure as described in Example 5 for 2-(chloromethyl)-5-methyl-3-(1H-pyrrol-1-yl)quinazolin-4(3H)-one, the title compound was synthesized from 2-bromo-6-[(chloro acetyl)amino]benzoic acid (Intermediate 4) and 1H-pyrrol-1-amine.

1H-NMR (400 MHz, DMSO-d6): δ 7.85 (1H, m), 7.73-7.79 (2H, m), 7.06 (2H, t), 6.26 (2H, t), 4.33 (2H, s).

ESMS: 337.88 (M−1), 339.88 (M+1)

### Example 7

### 2-(chloromethyl)-5-methyl-3-(1-phenylcyclopropyl)quinazolin-4(3H)-one (Intermediate 7)

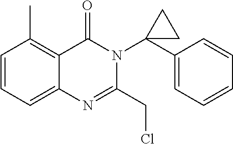 [SMILES: Cc1cccc2nc(CCl)n(C3(c4ccccc4)CC3)c(=O)c12]

Using the similar procedure as described in Example 5 for 2-(chloromethyl)-5-methyl-3-(1H-pyrrol-1-yl)quinazolin-4(3H)-one, the title compound was synthesized from 2-[(chloroacetyl) amino]-6-methylbenzoic acid (Intermediate 3) and 1-phenylcyclopropanamine.

1H-NMR (400 MHz, DMSO-d6): δ 7.69 (1H, t), 7.54 (1H, d), 7.32 (3H, t), 7.23 (1H, t), 6.97 (2H, d), 4.84 (1H, d), 4.66 (1H, d), 2.76 (3H, s), 1.79-1.88 (2H, m), 1.58-1.71 (2H, m).

ESMS: 325.02 (M+1)

### Example 8

### 2-[(4-amino-3-iodo-1H-pyrazolo[3,4-d]pyrimidin-1-yl)methyl]-5-methyl-3-(1H-pyrrol-1-yl)quinazolin-4(3H)-one (Intermediate 8)

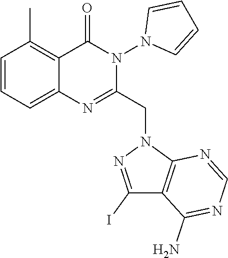 [SMILES: Cc1cccc2nc(Cn3nc(I)c4c(N)ncnc43)n(-n3cccc3)c(=O)c12]

To a stirred solution of 2-(chloromethyl)-5-methyl-3-(1H-pyrrol-1-yl)quinazolin-4(3H)-one (Intermediate 5) (28.5 gm, 104 mmol) and 3-iodo-1H-pyrazolo[3,4-d]pyrimidin-4-amine (26.1 gm, 100 mmol) in DMF (280 ml), potassium carbonate (43 gm, 311 mmol) was added at room temperature. The reaction mixture was stirred at room temperature for 4 to 6 h. The reaction mixture was poured into water. The solid thus precipitated was filtered, washed by diisopropyl ether and dried under vacuum to get 50 gm of title compound as light yellow solid.

1H-NMR (400 MHz, DMSO-d6): δ 8.18 (2H, s), 7.95 (1H, s), 7.64 (1H, t), 7.34 (1H, d), 7.25 (1H, d), 7.06 (2H, t), 6.20 (2H, t), 5.21 (2H, s), 2.89 (3H, s).

ESMS: 498.92 (M+)

### Example 9

### 2-[(4-amino-3-iodo-1H-pyrazolo[3,4-d]pyrimidin-1-yl)methyl]-5-bromo-3-(1H-pyrrol-1-yl)quinazolin-4(3H)-one (Intermediate 9)

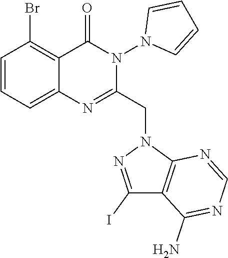 [SMILES: Nc1ncnc2c1c(I)nn2Cc1nc2cccc(Br)c2c(=O)n1-n1cccc1]

Using the similar procedure as described in Example 8 for 2-[(4-amino-3-iodo-1H-pyrazolo[3,4-d] pyrimidin-1-yl)methyl]-5-methyl-3-(1H-pyrrol-1-yl)quinazolin-4(3H)-one, title compound was synthesized from 5-bromo-2-(chloromethyl)-3-(1H-pyrrol-1-yl)quinazolin-4 (3H)-one (Intermediate 6) and 3-iodo-1H-pyrazolo[3,4-d]pyrimidin-4-amine.

1H-NMR (400 MHz, DMSO-d6): δ 8.18 (1H, s), 7.80 (1H, d), 7.62 (1H, t), 7.41 (1H, d), 7.12 (2H, t), 6.24 (2H, t), 5.24 (2H, s) (NH₂ protons exchanged due to moisture).

ESMS: 562.93 (M−1), 564.94 (M+1)

### Example 10

### 2-[(4-amino-3-iodo-1H-pyrazolo[3,4-d]pyrimidin-1-yl)methyl]-5-methyl-3-(1-phenyl cyclopropyl)quinazolin-4(3H)-one (Intermediate 10)

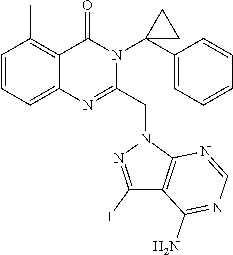 [SMILES: Cc1cccc2nc(Cn3nc(I)c4c(N)ncnc43)n(C3(c4ccccc4)CC3)c(=O)c12]

Using the similar procedure as described in Example 8 for 2-[(4-amino-3-iodo-1H-pyrazolo[3,4-d] pyrimidin-1-yl)methyl]-5-methyl-3-(1H-pyrrol-1-yl)quinazolin-4(3H)-one, title compound was synthesized from 2-(chloromethyl)-5-methyl-3-(1-phenylcyclopropyl)quinazolin-4(3H)-one (Intermediate 7) and 3-iodo-1H-pyrazolo[3,4-d]pyrimidin-4-amine.

1H-NMR (400 MHz, CDCl3): δ 8.26 (1H, s), 7.48 (1H, t), 7.13-7.35 (6H, m), 6.92 (1H, d), 6.09 (2H, s), 5.80 (1H, d), 5.60 (1H, d), 2.83 (3H, s), 2.01-2.08 (2H, m), 1.72-1.81 (2H, m).

ESMS:550.01 (M+1)

### EXAMPLES FOR PREPARATION OF COMPOUNDS OF FORMULA (I)

### Example 11

### 2-{[4-amino-3-(7-hydroxy-2,2-dimethyl-2,3-dihydro-1-benzofuran-4-yl)-1H-pyrazolo [3,4-d]pyrimidin-1-yl]methyl}-5-methyl-3-(1H-pyrrol-1-yl)quinazolin-4(3H)-one (Compound No. 2)

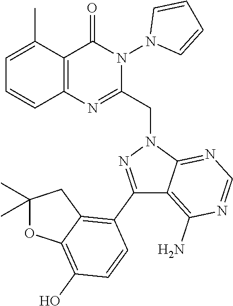 [SMILES: Cc1cccc2nc(Cn3nc(-c4ccc(O)c5c4CC(C)(C)O5)c4c(N)ncnc43)n(-n3cccc3)c(=O)c12]

To a stirred solution of 2-[(4-amino-3-iodo-1H-pyrazolo[3,4-d]pyrimidin-1-yl)methyl]-5-methyl-3-(1H-pyrrol-1-yl)quinazolin-4(3H)-one (Intermediate 8) (49.8 gm, 100 mmol) and 2,2-dimethyl-4-(4,4,5,5-tetramethyl-1,3,2-dioxaborolan-2-yl)-2,3-dihydro-1-benzofuran-7-ol (Intermediate 1) (40 gm, 138 mmol) in the mixture of dioxane (400 ml) and ethanol (100 ml), solution of K₃PO₄ (64 gm, 300 mmol) in water (100 ml) was added drop wise at room temperature. Nitrogen gas was purged for 30 min. at room temperature. Catalyst PdCl₂(PPh₃)₂ (14 gm, 20 mmol) was added to the reaction mixture under nitrogen atmosphere at room temperature. The reaction mixture was heated at 80° C. for 4 to 6 h. The reaction mixture was cooled to room temperature and poured into water (1500 ml). The solid obtained was filtered and dried under vacuum. The above solid was stirred in diisopropyl ether (1000 ml) for 1 h, filtered; solid was washed with ether and dried under vacuum. The solid was dissolved in ethyl acetate (3000 ml) and filtered through hyflow. Filtrate was concentrated to 200 ml and stirred at room temperature for 5 h. The solid obtained was filtered and dried under vacuum. The solid material further crystallized in 10% methanol in dichloromethane to get 30 gm of title compound as white crystalline solid.

1H-NMR (400 MHz, DMSO-d6): δ 9.45 (1H, s), 8.16 (1H, s), 7.66 (1H, t), 7.35 (1H, d), 7.29 (1H, d), 7.03 (2H, t), 6.83 (1H, d), 6.77 (1H, d), 6.19 (2H, t), 5.25 (2H, s), 3.02 (2H, s), 2.73 (3H, s), 1.36 (6H, s), (NH₂ protons exchanged due to moisture)

ESMS: 534.96 (M+)

### Example 12

### 2-{[4-amino-3-(7-hydroxy-2,2-dimethyl-2,3-dihydro-1-benzofuran-4-yl)-1H-pyrazolo [3,4-d]pyrimidin-1-yl]methyl}-5-bromo-3-(1H-pyrrol-1-yl)quinazolin-4(3H)-one (Compound No. 9)

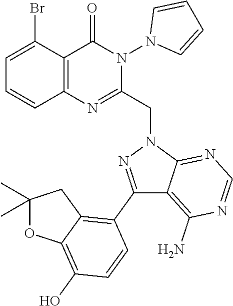 [SMILES: CC1(C)Cc2c(-c3nn(Cc4nc5cccc(Br)c5c(=O)n4-n4cccc4)c4ncnc(N)c34)ccc(O)c2O1]

To a stirred solution of 2-[(4-amino-3-iodo-1H-pyrazolo[3,4-d]pyrimidin-1-yl)methyl]-5-bromo-3-(1H-pyrrol-1-yl)quinazolin-4(3H)-one (Intermediate 9) (45 gm, 80 mmol) and 2,2-dimethyl-4-(4,4,5,5-tetramethyl-1,3,2-dioxaborolan-2-yl)-2,3-dihydro-1-benzofuran-7-ol (Intermediate 1) (32.48 gm, 112 mmol) in the mixture of dioxane (400 ml) and ethanol (100 ml), solution of K₃PO₄ (51 gm, 240 mmol) in water (100 ml) was added drop wise at room temperature. Nitrogen gas was purged for 30 min. at room temperature. Catalyst PdCl₂(PPh₃)₂ (11.2 gm, 16 mmol) was added to the reaction mixture under nitrogen atmosphere at room temperature. The reaction mixture was heated at 80° C. for 4 to 6 h. The reaction mixture was cooled to room temperature and poured into water (1500 ml). The solid obtained was filtered and dried under vacuum. The above solid was stirred in diisopropyl ether (1000 ml) for 1 h, filtered; solid was washed with ether and dried under vacuum. The solid was dissolved in ethyl acetate (3000 ml) and filtered through hyflow. Filtrate was concentrated to 200 ml and stirred at room temperature for 5 h to get 22 gm of title compound as white crystalline solid.

1H-NMR (400 MHz, DMSO-d6): δ 9.46 (1H, s), 8.17 (1H, s), 7.82 (1H, d), 7.65 (1H, t), 7.44 (1H, d), 7.07 (2H, t), 6.83 (1H, d), 6.78 (1H, d), 6.22 (2H, t), 5.25 (2H, s), 3.02 (2H, s), 1.37 (6H, s), (NH₂ protons exchanged due to moisture)

ESMS: 600.84 (M+1), 598.89 (M−1)

### Example 13

### 2-{[4-amino-3-(7-hydroxy-2,2-dimethyl-2,3-dihydro-1-benzofuran-4-yl)-1H-pyrazolo [3,4-d]pyrimidin-1-yl]methyl}-5-(1-methyl-1H-pyrazol-4-yl)-3-(1H-pyrrol-1-yl) quinazolin-4(3H)-one (Compound No. 44)

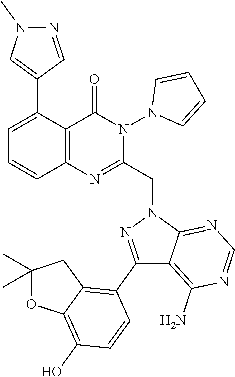 [SMILES: Cn1cc(-c2cccc3nc(Cn4nc(-c5ccc(O)c6c5CC(C)(C)O6)c5c(N)ncnc54)n(-n4cccc4)c(=O)c23)cn1]

To a stirred solution of 2-{[4-amino-3-(7-hydroxy-2,2-dimethyl-2,3-dihydro-1-benzo furan-4-yl)-1H-pyrazolo[3,4-d]pyrimidin-1-yl]methyl}-5-bromo-3-(1H-pyrrol-1-yl) quinazolin-4(3H)-one (Compound 9) (20 gm, 33 mmol) and 1-Methylpyrazole-4-boronic acid pinacol ester (1-Methyl-4-(4,4,5,5-tetramethyl-1,3,2-dioxaborolan-2-yl)-1H-pyrazole) (8.32 gm, 40 mmol) in the mixture of dioxane (150 ml) and ethanol (40 ml), solution of K₃PO₄ (21 gm, 100 mmol) in water (30 ml) was added drop wise at room temperature. Nitrogen gas was purged for 30 min. at room temperature. Catalyst PdCl₂(PPh₃)₂ (4.6 gm, 6.6 mmol) was added to the reaction mixture under nitrogen atmosphere at room temperature. The reaction mixture was heated at 80° C. for 4 to 6 h. The reaction mixture was cooled to room temperature and poured into water (500 ml). The solid obtained was filtered and dried under vacuum. The above solid was stirred in diisopropyl ether (500 ml) for 1 h, filtered; solid was washed with ether and dried under vacuum. The solid was dissolved in ethyl acetate (1000 ml) and filtered through hyflow. Filtrate was concentrated to 100 ml and stirred at room temperature for 5 h. The solid obtained was filtered and dried under vacuum. The solid material further crystallized in 10% methanol in dichloromethane to get 12 gm of title compound as white crystalline solid.

1H-NMR (400 MHz, DMSO-d6): δ 9.45 (1H, s), 8.17 (1H, s), 7.84 (1H, s), 7.74 (1H, t), 7.53 (1H, s), 7.41 (1H, d), 7.36 (1H, d), 7.02 (2H, s), 6.84 (1H, d), 6.78 (1H, d), 6.17 (2H, s), 5.27 (2H, s), 3.83 (3H, s), 3.04 (2H, s), 1.37 (6H, s), (NH2proton exchanged due to moisture).

ESMS: 601.20 (M+1)

### Example 14

### 2-{[4-amino-3-(7-hydroxy-2,2-dimethyl-2,3-dihydro-1-benzofuran-4-yl)-1H-pyrazolo [3,4-d]pyrimidin-1-yl]methyl}-5-methyl-3-(1-phenylcyclopropyl)quinazolin-4(3H)-one (Compound No. 8)

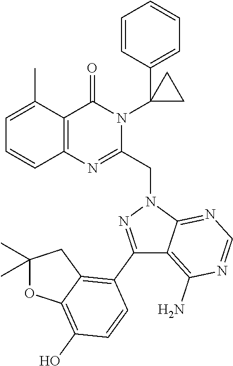 [SMILES: Cc1cccc2nc(Cn3nc(-c4ccc(O)c5c4CC(C)(C)O5)c4c(N)ncnc43)n(C3(c4ccccc4)CC3)c(=O)c12]

Using the similar procedure as described for 24[4-amino-3-(7-hydroxy-2,2-dimethyl-2,3-dihydro-1-benzofuran-4-yl)-1H-pyrazolo[3,4-d]pyrimidin-1-yl]methyl}-5-methyl-3-(1H-pyrrol-1-yl)quinazolin-4(3H)-one (Compound 2), title compound can be synthesized from 2-[(4-amino-3-iodo-1H-pyrazolo[3,4-d]pyrimidin-1-yl)methyl]-5-methyl-3-(1-phenylcyclopropyl) quinazolin-4(3H)-one (Intermediate 10) and 2,2-dimethyl-4-(4,4,5,5-tetramethyl-1,3,2-dioxaborolan-2-yl)-2,3-dihydro-1-benzofuran-7-ol (Intermediate 1).

1H-NMR (400 MHz, DMSO-d6): δ 9.44 (1H, s), 8.18 (1H, s), 7.54 (1H, t), 7.24-7.30 (3H, m), 7.20 (1H, t), 7.10 (1H, d), 7.04 (2H, d), 6.81 (1H, d), 6.77 (1H, d), 5.88 (1H, d), 5.36 (1H, d), 2.96 (2H, q), 2.73 (3H, s), 1.94-2.00 (2H, m), 1.71-1.73 (1H, m), 1.64-1.66 (1H, m), 1.36 (6H, s), (NH₂ protons exchanged due to moisture)

ESMS: 586.10 (M+1)

The following representative compounds of the present invention were prepared in analogous manner by using the synthetic schemes as described above:

TABLE 1

| Comp. | | |
|---|---|---|
| No. | ¹H-NMR (400 MHz, DMSO-d₆) | MASS |
| 1 | δ 9.57 (1H, s), 8.17 (1H, s), 7.74 (1H, t), 7.62 (1H, m), | 525.20 |
| | 7.39 (1H, m), 7.09 (3H, s), 6.76 (1H, m), 6.23 (2H, s), | (M+) |
| | 5.26 (2H, s), 2.81-2.86 (4H, m), 1.95 (2H, s), (NH₂ | |
| | protons exchanged due to moisture) | |
| 3 | δ 9.47 (1H, s), 8.17 (1H, s), 7.74 (1H, t), 7.61 (1H, d), | 554.87 |
| | 7.40 (1H, d), 7.08 (2H, t), 6.83 (1H, d), 6.78 (1H, d), | (M+) |
| | 6.23 (2H, t), 5.25 (2H, s), 3.02 (2H, s), 1.37 (6H, s), (NH₂ | |
| | protons exchanged due to moisture) | |
| 4 | δ 9.45 (1H, s), 8.34 (1H, s), 7.94 (1H, s), 7.79-7.87 (2H, | 605.82 |
| | quintet), 7.66 (2H, q), 7.35-7.41 (2H, m), 7.24 (1H, t), | (M − 1) |
| | 6.77 (2H, s), 5.41 (2H, q), 2.96 (2H, s), 1.36 (6H, s), (NH₂ | |
| | protons exchanged due to moisture) | |
| 5 | δ 9.44 (1H, s), 8.19 (1H, s), 7.63 (1H, t), 7.52 (1H, d), | 605.88 |
| | 7.30 (2H, t), 7.22 (2H, d), 7.06 (2H, d), 6.82 (1H, d), | (M − 1) |
| | 6.77 (1H, d), 5.91 (1H, d), 5.36 (1H, d), 2.96 (2H, d), | |
| | 1.98-2.03 (2H, m), 1.71-1.73 (1H, m), 1.64 (1H, m), 1.36 (6H, | |
| | s), (NH₂ protons exchanged due to moisture) | |
| 6 | δ 9.46 (1H, s), 8.17 (1H, s), 8.14 (1H, d), 7.92 (1H, s), | 556.11 |
| | 7.78-7.80 (1H, m), 7.76 (1H, t), 7.66 (1H, d), 7.44 (1H, | (M + 1) |
| | d), 7.16 (1H, bs), 6.84 (1H, d), 6.78 (1H, d), 6.56 (1H, t), | |
| | 5.26 (2H, s), 3.04 (2H, s), 1.37-1.38 (6H, s) | |
| 7 | δ 9.46 (1H, s), 8.16 (1H, s), 8.11 (1H, d), 7.68-7.73 (2H, | 536.02 |
| | m), 7.39 (1H, d), 7.31 (1H, d), 6.84 (1H, d), 6.78 (1H, d), | (M + 1) |
| | 6.53 (1H, t), 5.24 (2H, q), 3.04 (2H, s), 2.72 (3H, s), | |
| | 1.37 (6H, s), (NH₂ protons exchanged due to moisture) | |
| 10 | δ 9.49 (1H, s), 8.58 (2H, s), 8.21 (1H, s), 7.85-7.89 (2H, | 598.14 |
| | m), 7.55 (1H, d), 7.48-7.51 (1H, m), 7.41 (1H, d), | (M + 1) |
| | 7.02 (2H, s), 6.85 (1H, d), 6.80 (1H, d), 6.16 (2H, s), 5.31 (2H, | |
| | s), 3.06 (2H, s), 1.40 (6H, s), (NH₂ protons exchanged | |
| | due to moisture) | |
| 11 | δ 9.45 (1H, s), 8.15 (1H, s), 7.73 (1H, t), 7.41 (2H, d), | 564.18 |
| | 6.81 (1H, d), 6.77 (1H, d), 6.02 (1H, s), 5.33 (2H, s), | (M + 1) |
| | 2.99 (2H, s), 2.72 (3H, s), 2.11 (3H, s), 1.98 (3H, s), 1.36 (6H, | |
| | s), (NH₂ protons exchanged due to moisture) | |
| 12 | δ 9.45 (1H, s), 8.16 (1H, s), 7.81 (1H, t), 7.67 (1H, d), | 584.07 |
| | 7.53 (1H, d), 6.81 (1H, d), 6.77 (1H, d), 6.05 (1H, s), | (M+) |
| | 5.34 (2H, q), 2.99 (2H, s), 2.10 (3H, s), 2.01 (3H, s), 1.36 (6H, | |
| | s), (NH₂ protons exchanged due to moisture) | |
| 13 | δ 9.45 (1H, s), 8.18 (1H, s), 7.74 (1H, d), 7.54 (1H, t), | 650.11 |
| | 7.26-7.32 (3H, m), 7.21-7.23 (1H, m), 7.05 (2H, d), | (M+), |
| | 6.82 (1H, d), 6.77 (1H, d), 5.91 (1H, d), 5.37 (1H, d), | 652.07 |
| | 2.95 (2H, q), 1.98-2.04 (2H, m), 1.63-1.74 (2H, m), 1.36 (6H, | (M + 2) |
| | s), (NH₂ protons exchanged due to moisture) | |
| 14 | δ 9.47 (1H, s), 8.18 (1H, s), 7.78 (1H, t), 7.39-7.46 (4H, | 603.19 |
| | m), 7.10 (1H, d), 7.03 (2H, s), 6.78-6.87 (2H, m), | (M + 1) |
| | 6.17 (2H, s), 5.28 (2H, s), 3.05 (2H, s), 1.39 (6H, s), (NH₂ | |
| | protons exchanged due to moisture) | |
| 15 | δ 9.46 (1H, s), 8.17 (1H, s), 7.80 (1H, t), 7.61 (1H, d), | 601.09 |
| | 7.44 (1H, d), 7.04 (2H, t), 6.84 (1H, d), 6.78 (1H, d), | (M + 1) |
| | 6.62 (1H, d), 6.15-6.19 (3H, m), 5.26 (2H, s), 3.03 (2H, s), | |
| | 2.28 (3H, s), 1.37 (6H, s), (NH₂ protons exchanged due | |
| | to moisture) | |
| 16 | δ 9.57 (1H, s), 8.87 (1H, s), 8.73 (1H, d), 8.34 (1H, s), | 649.14 |
| | 8.31 (1H, d), 7.83 (2H, t), 7.41-7.47 (2H, m), | (M + 1) |
| | 7.23-7.26 (2H, m), 7.17 (1H, t), 6.97 (2H, d), 6.82 (2H, q), | |
| | 5.99 (1H, d), 5.46 (1H, d), 2.99 (2H, q), 1.99-2.03 (2H, m), | |
| | 1.66-1.70 (1H, m), 1.54-1.60 (1H, m), 1.39-1.40 (6H, d), | |
| | (NH₂ protons exchanged due to moisture) | |
| 17 | δ 9.45 (1H, s), 8.19 (1H, s), 7.69 (1H, t), 7.54 (1H, d), | 652.19 |
| | 7.24-7.29 (3H, m), 7.17-7.20 (1H, m), 7.01 (2H, d), | (M + 1) |
| | 6.83 (1H, d), 6.78 (1H, d), 6.62 (1H, d), 6.15 (1H, s), | |
| | 5.90 (1H, d), 5.39 (1H, d), 2.97 (2H, s), 2.28 (3H, s), | |
| | 1.98-2.05 (2H, m), 1.62-1.68 (2H, m), 1.36 (6H, s), (NH₂ protons | |
| | exchanged due to moisture) | |
| 18 | δ 9.47 (1H, s), 8.53 (2H, d), 8.18 (1H, s), 7.85 (1H, t), | 598.00 |
| | 7.54 (1H, d), 7.31-7.35 (3H, m), 7.02 (2H, t), 6.83 (1H, | (M + 1) |
| | d), 6.79 (1H, d), 6.16 (2H, t), 5.29 (2H, s), 3.06 (2H, s), | |
| | 1.40 (6H, s), (NH₂ protons exchanged due to moisture) | |
| 19 | δ 9.46 (1H, s), 8.17 (1H, s), 7.75 (1H, t), 7.60 (1H, d), | 575.11 |
| | 7.42 (1H, d), 7.07 (2H, t), 6.83 (1H, d), 6.78 (1H, d), | (M + 1), |
| | 6.22 (2H, t), 5.33 (1H, t), 5.26 (2H, s), 4.32 (2H, d), 3.02 (2H, | 576.11 |
| | s), 1.37 (6H, s), (NH₂ protons exchanged due to moisture) | (M + 2) |
| 20 | δ 9.46 (1H, s), 8.17 (1H, s), 7.74-7.78 (1H, m), 7.68 (1H, | 545.06 |
| | d), 7.47 (1H, d), 7.07 (2H, t), 6.77-6.85 (2H, m), | (M + 1) |
| | 6.22 (2H, t), 5.26 (2H, s), 4.50 (1H, s), 3.02 (2H, s), 1.37 (6H, | |
| | s), (NH₂ protons exchanged due to moisture) | |
| 21 | δ 9.45 (1H, s), 8.52 (2H, d), 8.20 (1H, s), 7.74 (1H, s), | 649.15 |
| | 7.27-7.37 (3H, m), 7.24-7.26 (3H, m), 7.17 (1H, t), | (M + 1) |
| | 6.98 (2H, d), 6.83 (1H, d), 6.78 (1H, d), 5.94 (1H, d), 5.37 (1H, | |
| | d), 2.99 (2H, q), 1.95-1.99 (2H, m), 1.60 (2H, m), | |
| | 1.38 (6H, s), (NH₂ protons exchanged due to moisture) | |
| 22 | δ 9.47 (1H, s), 8.19 (1H, s), 7.86 (1H, t), 7.57 (1H, d), | 601.07 |
| | 7.37-7.41 (2H, m), 7.07 (2H, bs), 6.85 (1H, d), 6.79 (1H, | (M + 1) |
| | d), 6.17-6.19 (3H, m), 5.29 (2H, s), 3.45 (3H, s), | |
| | 3.06 (2H, s), 1.40 (6H, s), (NH₂ protons exchanged due to | |
| | moisture) | |
| 23 | δ 9.46 (1H, s), 8.17 (1H, s), 7.75-7.78 (2H, m), 7.61 (1H, | 587.05 |
| | s), 7.42 (2H, t), 7.03 (2H, s), 6.84 (1H, d), 6.78 (1H, d), | (M + 1) |
| | 6.58 (1H, s), 6.17 (2H, s), 5.28 (2H, s), 3.04 (2H, s), | |
| | 1.38 (6H, s), (NH₂ protons exchanged due to moisture) | |
| 24 | δ 9.45 (1H, s), 8.18 (1H, s), 7.64 (1H, t), 7.53 (1H, d), | 626.08 |
| | 7.20-7.31 (4H, m), 7.03 (2H, d), 6.81 (1H, d), 6.77 (1H, | (M + 1) |
| | d), 5.90 (1H, d), 5.37 (1H, d), 5.32 (1H, t), 4.32 (2H, d), | |
| | 2.95 (2H, q), 1.96-2.05 (2H, m), 1.63-1.75 (2H, m), | |
| | 1.35 (6H, s), (NH₂ protons exchanged due to moisture) | |
| 25 | δ 9.46 (1H, s), 8.20 (1H, s), 7.66 (1H, m), 7.43 (2H, m), | 654.12 |
| | 7.26 (4H, m), 7.18 (1H, m), 7.10 (1H, s), 6.99 (2H, m), | (M + 1) |
| | 6.80 (2H, m), 5.90 (1H, d), 5.39 (1H, d), 2.99 (2H, s), | |
| | 1.99 (2H, m), 1.64 (2H, m), 1.37 (6H, s), (NH₂ protons | |
| | exchanged due to moisture) | |
| 26 | δ 9.45 (1H, s), 8.19 (1H, s), 7.67-7.72 (1H, m), | 590.09 |
| | 7.21-7.32 (4H, m), 7.05-7.12 (3H, m), 6.82 (1H, d), 6.77 (1H, d), | (M + 1) |
| | 5.92 (1H, d), 5.36 (1H, d), 2.96 (2H, q), 1.99-2.01 (2H, | |
| | m), 1.70 (2H, m), 1.37 (6H, s), (NH₂ protons exchanged | |
| | due to moisture) | |
| 27 | δ 9.45 (1H, s), 8.17 (1H, s), 7.95 (1H, s), 7.66 (1H, d), | 535.11 |
| | 7.39 (1H, d), 7.09 (2H, s), 6.83 (1H, d), 6.78 (1H, d), | (M + 1) |
| | 6.22 (2H, s), 5.25 (2H, s), 3.02 (2H, s), 2.47 (3H, d), 1.37 (6H, | |
| | s), (NH₂ protons exchanged due to moisture) | |
| 28 | δ 9.47 (1H, s), 8.39 (1H, t), 8.17 (1H, s), 7.74 (1H, t), | 616.13 |
| | 7.59 (1H, d), 7.42 (1H, d), 7.08 (2H, s), 6.83 (1H, d), 6.78 (1H, | (M + 1) |
| | d), 6.22 (2H, s), 5.26 (2H, s), 4.12 (2H, d), 3.03 (2H, s), | |
| | 1.83 (3H, s), 1.37 (6H, s), (NH₂ protons exchanged due | |
| | to moisture) | |
| 29 | δ 9.45 (1H, s), 8.60 (1H, t), 8.17 (1H, s), 7.74 (1H, t), | 642.23 |
| | 7.60 (1H, d), 7.42 (1H, d), 7.08 (2H, t), 6.83 (1H, d), 6.78 (1H, | (M + 1) |
| | d), 6.22 (2H, t), 5.26 (2H, s), 4.16 (2H, d), 3.02 (2H, s), | |
| | 1.54-1.60 (1H, m), 1.37 (6H, s), 0.65-0.68 (4H, m), (NH₂ | |
| | protons exchanged due to moisture) | |
| 30 | δ 9.45 (1H, s), 8.20 (1H, t), 7.76 (1H, s), 7.37-7.42 (2H, | 652.31 |
| | m), 7.25-7.32 (3H, m), 7.18 (1H, t), 7.00 (2H, d), | (M + 1) |
| | 6.83 (1H, d), 6.78 (1H, d), 6.17 (1H, d), 5.94 (1H, d), 5.39 (1H, | |
| | d), 3.42 (3H, s), 2.98 (2H, q), 2.05 (1H, m), | |
| | 1.95-1.99 (1H, m), 1.65 (1H, m), 1.52 (1H, m), 1.37 (6H, s), (NH₂ | |
| | protons exchanged due to moisture) | |
| 31 | δ 9.44 (1H, s), 8.18 (1H, s), 7.62-7.64 (1H, m), 7.53 (1H, | 640.16 |
| | d), 7.25 (1H, d), 7.15-7.17 (1H, m), 7.01 (1H, d), | (M + 1) |
| | 6.89 (1H, s), 6.76-6.81 (3H, m), 5.89 (1H, d), 5.39 (1H, d), | |
| | 5.31 (1H, t), 4.32 (2H, d), 2.96 (2H, s), 2.23 (3H, s), | |
| | 2.03 (1H, m), 1.90 (1H, m), 1.77 (1H, m), 1.60 (1H, m), | |
| | 1.34 (6H, s), (NH₂ protons exchanged due to moisture) | |
| 32 | δ 9.45 (1H, s), 8.19 (1H, s), 7.95 (1H, d), 7.66 (1H, d), | 535.17 |
| | 7.44 (1H, t), 7.18-7.21 (2H, m), 6.81 (1H, d), 6.78 (1H, d), | (M + 1) |
| | 6.28 (2H, s), 5.27 (2H, s), 3.02 (2H, s), 2.06 (3H, s), | |
| | 1.39 (6H, s), (NH₂ protons exchanged due to moisture) | |
| 33 | δ 9.45 (1H, s), 8.19 (1H, s), 7.76 (1H, t), 7.38-7.42 (2H, | 666.20 |
| | m), 7.32 (1H, d), 7.14 (1H, t), 6.99 (1H, d), 6.83 (2H, d), | (M + 1) |
| | 6.78 (2H, d), 6.17 (1H, s), 5.92 (1H, d), 5.41 (1H, d), | |
| | 3.42 (3H, s), 2.99 (2H, s), 2.21 (3H, s), 2.03 (1H, m), 1.92 (1H, | |
| | m), 1.66 (1H, m), 1.49 (1H, m), 1.36 (6H, s), (NH₂ | |
| | protons exchanged due to moisture) | |
| 34 | δ 9.81 (1H, bs), 9.44 (1H, s), 8.17 (1H, s), 7.79 (1H, m), | 630.21 |
| | 7.71 (1H, m), 7.49 (1H, d), 7.09 (2H, s), 6.83 (1H, d), | (M + 1) |
| | 6.79 (1H, d), 6.24 (2H, s), 5.26 (2H, s), 4.33 (2H, bs), | |
| | 3.20 (4H, m), 3.04 (2H, s), 1.39 (6H, s), 1.20 (6H, m), | |
| | (NH proton exchanged due to moisture) | |
| 35 | δ 9.45 (1H, s), 8.17 (1H, s), 7.74 (1H, t), 7.63 (1H, d), | 644.23 |
| | 7.42 (1H, d), 7.08 (2H, t), 6.83 (1H, d), 6.77 (1H, d), | (M + 1) |
| | 6.22 (2H, t), 5.23 (2H, s), 3.58 (4H, t), 3.52 (2H, s), 3.02 (2H, | |
| | s), 2.50 (4H, t), 1.37 (6H, s), (NH₂ proton exchanged due | |
| | to moisture) | |
| 36 | δ 8.13 (1H, s), 7.65 (1H, t), 7.22 (1H, d), 7.09 (1H, d), | 561.21 |
| | 6.93 (2H, s), 6.80 (2H, d), 6.18 (2H, s), 5.26 (2H, s), | (M + 1) |
| | 3.24 (1H, s), 2..95 (2H, s), 1.31 (6H, s), 0.99 (2H, d), 0.73 (2H, | |
| | d), (NH₂ and OH protons exchanged in D₂O exchange) | |
| | (DMSO-d₆ and D₂O exchange) | |
| 37 | δ 9.45 (1H, s), 8.19 (1H, s), 7.64 (1H, t), 7.53 (1H, d), | 640.23 |
| | 7.24 (1H, d), 7.09 (2H, d), 6.92 (2H, d), 6.82 (1H, d), | (M + 1) |
| | 6.78 (1H, d), 5.90 (1H, d), 5.38 (1H, d), 5.32 (1H, t), 4.33 (2H, | |
| | d), 2.96 (2H, q), 2.25 (3H, s), 1.99-2.00 (1H, m), | |
| | 1.89-1.91 (1H, m), 1.69-1.70 (1H, m), 1.58-1.60 (1H, m), | |
| | 1.36 (6H, s), (NH₂ proton exchanged due to moisture) | |
| 38 | δ 9.45 (1H, s), 8.18 (1H, s), 7.64 (1H, t), 7.54 (1H, d), | 681.32 |
| | 7.30 (2H, t), 7.21 (2H, t), 7.05 (2H, d), 6.82 (1H, d), | (M + 1) |
| | 6.77 (1H, d), 5.90 (1H, d), 5.36 (1H, d), 3.70 (2H, m), | |
| | 2.95 (2H, q), 2.67-2.73 (4H, m), 1.97-2.03 (2H, m), | |
| | 1.71-1.73 (1H, m), 1.63 (1H, m), 1.36 (6H, s), 1.02-1.04 (6H, m), | |
| | (NH₂ proton exchanged due to moisture) | |
| 39 | δ 9.47 (1H, s), 8.19 (1H, s), 7.68 (1H, t), 7.37 (1H, d), | 563.15 |
| | 7.27 (1H, d), 6.84 (1H, d), 6.79 (1H, d), 5.95 (2H, s), | (M + 1) |
| | 5.14 (2H, s), 3.03 (2H, s), 2.75 (3H, s), 2.06 (6H, s), 1.38 (6H, | |
| | s), (NH₂ proton exchanged due to moisture) | |
| 40 | δ 9.47 (1H, s), 8.20 (1H, s), 7.75 (1H, t), 7.38-7.41 (3H, | 666.20 |
| | m), 7.31 (1H, d), 7.06 (2H, d), 6.87-6.89 (2H, m), | (M + 1) |
| | 6.80-6.84 (2H, m), 5.93 (1H, d), 5.39 (1H, d), 3.42 (3H, s), | |
| | 2.98 (2H, q), 2.23 (3H, s), 2.00 (1H, m), 1.91 (1H, m), | |
| | 1.61 (1H, m), 1.48 (1H, m), 1.38 (6H, s), (NH₂ proton | |
| | exchanged due to moisture) | |
| 41 | δ 9.47 (1H, s), 8.20 (1H, s), 7.89 (1H, t), 7.58 (1H, d), | 629.24 |
| | 7.44 (1H, d), 7.39 (1H, d), 6.85 (1H, d), 6.79 (1H, d), | (M + 1) |
| | 6.22 (1H, d), 5.92 (2H, s), 5.19 (2H, s), 3.44 (3H, s), 3.05 (2H, | |
| | s), 2.03 (6H, s), 1.40 (6H, s), (NH₂ proton exchanged due | |
| | to moisture) | |
| 42 | δ 9.46 (1H, s), 8.19 (1H, s), 7.77 (1H, t), 7.63 (1H, d), | 603.14 |
| | 7.41 (1H, d), 6.84 (1H, d), 6.78 (1H, d), 5.96 (2H, s), | (M + 1) |
| | 5.34 (1H, t), 5.16 (2H, s), 4.33 (2H, d), 3.03 (2H, s), 2.06 (6H, | |
| | s), 1.38 (6H, s), (NH₂ proton exchanged due to moisture) | |
| 43 | δ 9.46 (1H, s), 8.19 (1H, s), 7.70 (1H, t), 7.55 (1H, d), | 613.03 |
| | 7.33 (1H, d), 6.83 (1H, d), 6.78 (1H, d), 5.97 (2H, s), | (M + 1) |
| | 5.11 (2H, s), 3.02 (2H, s), 2.06 (6H, s), 1.53-1.60 (1H, m), | |
| | 1.38 (6H, s), 0.88-0.93 (2H, m), 0.74-0.78 (2H, m), (NH₂ | |
| | proton exchanged due to moisture) | |
| 45 | δ 9.45 (1H, s), 8.17 (1H, s), 7.89 (1H, s), 7.74 (1H, t), | 615.19 |
| | 7.55 (1H, s), 7.43 (1H, d), 7.35 (1H, d), 7.03 (2H, s), | (M + 1) |
| | 6.84 (1H, d), 6.78 (1H, d), 6.17 (2H, s), 5.26 (2H, s), 4.12 (2H, | |
| | q), 3.04 (2H, s), 1.37-1.39 (9H, m), (NH₂ proton | |
| | exchanged due to moisture) | |
| 48 | δ 9.45 (1H, s), 8.17 (1H, s), 7.69 (1H, t), 7.52 (1H, d), | 585.31 |
| | 7.34 (1H, d), 7.06 (2H, s), 6.83 (1H, d), 6.77 (1H, d), | (M + 1) |
| | 6.22 (2H, s), 5.22 (2H, s), 3.01 (2H, s), 1.55 (1H, m), 1.37 (6H, | |
| | s), 0.88-0.91 (2H, m), 0.73-0.75 (2H, m), (NH₂ proton | |
| | exchanged due to moisture) | |
| 49 | δ 9.45 (1H, s), 8.18 (1H, s), 7.73 (1H, t), 7.55 (1H, d), | 603.21 |
| | 7.39 (1H, d), 7.09 (2H, s), 6.83 (1H, d), 6.78 (1H, d), | (M + 1) |
| | 6.23 (2H, s), 5.42 (1H, s), 5.22 (2H, s), 3.02 (2H, s), 1.44 (6H, | |
| | s), 1.37 (6H, s), (NH₂ proton exchanged due to moisture) | |
| 50 | δ 9.45 (1H, s), 8.25 (1H, bs), 7.62 (1H, t), 7.48 (1H, d), | 668.27 |
| | 7.21 (1H, d), 7.10 (2H, d), 6.92 (2H, d), 6.82 (1H, d), | (M + 1) |
| | 6.78 (1H, d), 5.90 (1H, d), 5.41 (1H, s), 5.35 (1H, d), 2.96 (2H, | |
| | q), 2.25 (3H, s), 1.96-1.99 (1H, m), 1.88-1.93 (1H, m), | |
| | 1.71-1.74 (1H, m), 1.61-1.63 (1H, m), 1.46 (6H, s), | |
| | 1.36 (6H, s), (NH₂ proton exchanged due to moisture) | |
| 51 | δ 9.44 (1H, s), 8.18 (1H, s), 7.57 (1H, t), 7.43 (1H, d), | 650.31 |
| | 7.16 (1H, d), 7.09 (2H, d), 6.91 (2H, d), 6.81 (1H, d), | (M + 1) |
| | 6.77 (1H, d), 5.87 (1H, d), 5.35 (1H, d), 2.95 (2H, q), 2.25 (3H, | |
| | s), 1.97 (1H, m), 1.88-1.90 (1H, m), 1.69-1.70 (1H, m), | |
| | 1.53-1.58 (2H, m), 1.35 (6H, s), 0.87-0.90 (2H, m), | |
| | 0.75-0.76 (2H, m), (NH₂ proton exchanged due to moisture) | |
| 52 | δ 8.19 (1H, s), 8.15 (1H, d), 7.84 (1H, t), 7.59 (1H, t), | 549.19 |
| | 7.49 (1H, d), 7.11 (2H, m), 6.93-6.98 (2H, m), 6.23 (2H, | (M + 1) |
| | t), 5.28 (2H, s), 4.08 (2H, q), 3.05 (2H, s), 1.37 (6H, s), | |
| | 1.32-1.36 (3H, m), (NH₂ proton exchanged due to | |
| | moisture) | |
| 53 | δ 9.44 (1H, s), 8.23 (1H, bs), 7.62 (1H, t), 7.48 (1H, d), | 654.18 |
| | 7.31 (2H, t), 7.21-7.23 (2H, m), 7.04 (2H, d), 6.82 (1H, | (M + 1) |
| | d), 6.77 (1H, d), 5.90 (1H, d), 5.41 (1H, s), 5.34 (1H, d), | |
| | 2.96 (2H, q), 1.94-2.03 (2H, m), 1.76-1.78 (1H, m), | |
| | 1.65 (1H, m), 1.46 (6H, s), 1.37 (6H, s), (NH₂ proton | |
| | exchanged due to moisture) | |
| 54 | δ 9.44 (1H, s), 8.18 (1H, s), 7.58 (1H, t), 7.44 (1H, d), | 636.21 |
| | 7.30 (2H, t), 7.16-7.23 (2H, m), 7.03 (2H, d), 6.81 (1H, | (M + 1) |
| | d), 6.77 (1H, d), 5.89 (1H, d), 5.35 (1H, d), 2.95 (2H, q), | |
| | 1.98-2.03 (1H, m), 1.93-1.96 (1H, m), 1.74-1.78 (1H, m), | |
| | 1.63-1.66 (1H, m), 1.52-1.59 (1H, m), 1.36 (6H, s), | |
| | 0.87-0.91 (2H, m), 0.73-0.77 (2H, m), (NH₂ proton exchanged | |
| | due to moisture) | |
| 55 | δ 8.34 (1H, s), 7.40-7.44 (2H, s), 7.26 (1H, m), | 620.13 |
| | 7.12-7.17 (5H, m), 6.95 (1H, d), 6.87 (1H, d), 6.12 (1H, bs), | (M+) |
| | 5.54 (2H, s), 3.10 (2H, s), 2.41 (3H, s), 2.30 (1H, m), 2.16 (1H, | |
| | m), 1.86 (1H, m), 1.59 (1H, m), 1.45 (6H, s), (NH proton | |
| | exchanged due to moisture) (in CDCl₃) | |
| 56 | δ 8.20 (1H, s), 7.55 (1H, s), 7.28 (1H, s), 7.12 (3H, m), | 600.20 |
| | 7.03 (2H, m), 6.82 (2H, s), 6.01 (1H, d), 5.35 (1H, d), | (M + 1) |
| | 2.93 (2H, s), 2.76 (3H, s), 2.25 (3H, s), 1.78 (4H, s), | |
| | 1.33 (6H, s), (NH₂ and OH proton exchanged due to moisture) | |
| | (in DMSO-d₆ + D₂O) | |
| 57 | δ 8.11 (1H, s), 7.56-7.64 (2H, s), 7.30 (1H, d), 6.82 (1H, | 685.21 |
| | d), 6.73 (1H, d), 5.91 (2H, s), 5.09 (2H, s), 3.51 (2H, s), | (M + 1) |
| | 2.99 (2H, s), 2.76 (4H, m), 2.47 (4H, m), 2.20 (3H, s), | |
| | 2.03 (6H, s), 1.34 (6H, m), (NH₂ and OH proton | |
| | exchanged due to moisture) (in MeOD) | |
| 58 | δ 9.47 (1H, s), 8.17 (1H, s), 7.72-7.76 (2H, m), 7.65 (1H, | 657.11 |
| | d), 7.43 (1H, d), 7.08 (2H, t), 6.83 (1H, d), 6.78 (1H, d), | (M + 1) |
| | 6.22 (2H, t), 5.23 (2H, s), 3.64 (2H, s), 3.16 (2H, s), | |
| | 3.09 (1H, s), 3.02 (2H, s), 2.69-2.73 (3H, m), 1.37 (6H, s), | |
| | (NH₂ proton exchanged due to moisture) | |
| 59 | δ 9.46 (1H, s), 8.20 (1H, s), 7.73 (1H, t), 7.40 (1H, d), | 666.16 |
| | 7.29-7.34 (2H, m), 7.05-7.15 (4H, m), 6.84 (1H, d), | (M + 1) |
| | 6.78 (1H, d), 6.17 (1H, s), 6.06 (1H, d), 5.35 (1H, d), 3.44 (3H, | |
| | s), 2.97 (2H, s), 2.33 (3H, s), 1.82 (2H, m), 1.69 (2H, m), | |
| | 1.36 (6H, s), (NH₂ proton exchanged due to moisture) | |
| 60 | δ 9.44 (1H, s), 8.20 (1H, s), 7.48-7.53 (2H, m), | 592.06 |
| | 7.22-7.24 (2H, m), 7.06 (1H, d), 6.93 (1H, q), 6.84 (1H, d), | (M + 1) |
| | 6.78 (1H, d), 5.96 (1H, d), 5.49 (1H, d), 2.96 (2H, q), 2.74 (3H, | |
| | s), 1.92-1.97 (1H, m), 1.81-1.87 (1H, m), 1.67-1.72 (1H, | |
| | m), 1.52-1.58 (1H, m), 1.36-1.37 (6H, d), (NH₂ proton | |
| | exchanged due to moisture) | |
| 62 | δ 9.47 (1H, s), 8.19 (1H, s), 7.86 (1H, s), 7.76 (1H, t), | 629.14 |
| | 7.54 (1H, s), 7.44 (1H, d), 7.34 (1H, d), 6.85 (1H, d), | (M + 1) |
| | 6.79 (1H, d), 5.93 (2H, s), 5.15 (2H, s), 3.84 (3H, s), | |
| | 3.04 (2H, s), 2.05 (6H, s), 1.38 (6H, s), (NH₂ proton exchanged | |
| | due to moisture) | |
| 63 | δ 9.46 (1H, s), 8.18 (1H, s), 7.94-8.02 (2H, m), 7.77 (1H, | 588.96 |
| | d), 7.09 (2H, t), 6.84 (1H, d), 6.78 (1H, d), 6.23 (2H, t), | (M+) |
| | 5.30 (2H, s), 3.03 (2H, s), 1.37 (6H, s), (NH₂ proton | |
| | exchanged due to moisture) | |
| 64 | δ 9.46 (1H, s), 8.18 (1H, s), 7.73-7.81 (3H, m), 7.44 (1H, | 625.14 |
| | d), 7.35 (1H, s), 7.12 (2H, s), 6.84 (1H, d), 6.78 (1H, d), | (M + 1) |
| | 6.24 (2H, s), 5.24 (2H, s), 3.73 (3H, s), 3.03 (2H, s), | |
| | 1.38 (6H, s), (NH₂ proton exchanged due to moisture) | |
| 65 | δ 9.44 (1H, s), 8.19 (1H, s), 7.55 (1H, t), 7.43 (1H, d), | 650.39 |
| | 7.06-7.18 (5H, m), 6.82 (1H, d), 6.77 (1H, d), 6.01 (1H, | (M + 1) |
| | d), 5.32 (1H, d), 2.94 (2H, s), 2.27 (4H, m), 1.81 (3H, s), | |
| | 1.56-1.59 (1H, m), 1.34 (6H, s), 0.90-0.93 (2H, m), | |
| | 0.78-0.79 (2H, m), (NH₂ proton exchanged due to moisture) | |
| 67 | δ 9.47 (1H, s), 8.20 (1H, s), 7.76-7.83 (3H, m), 7.43 (1H, | 653.24 |
| | d), 7.35 (1H, s), 6.84 (1H, d), 6.78 (1H, d), 5.98 (2H, s), | (M + 1) |
| | 5.11 (2H, s), 3.74 (3H, s), 3.04 (2H, s), 2.09 (6H, s), | |
| | 1.38 (6H, s), (NH₂ proton exchanged due to moisture) | |
| 68 | δ 9.46 (1H, s), 8.19 (1H, s), 7.74-7.76 (2H, m), 7.68 (1H, | 685.28 |
| | d), 7.41 (1H, d), 6.80 (2H, dd), 5.96 (2H, s), 5.11 (2H, | (M + 1) |
| | s), 3.65 (2H, s), 3.16 (2H, m), 3.09 (2H, s), 3.02 (2H, s), | |
| | 2.71 (2H, t), 2.06 (6H, s), 1.37 (6H, s), (NH₂ proton | |
| | exchanged due to moisture) | |
| 69 | δ 9.45 (1H, s), 8.20 (1H, s), 7.85 (1H, s), 7.60 (1H, t), | 655.95 |
| | 7.53 (1H, s), 7.46-7.48 (1H, m), 7.30 (1H, d), 7.22 (1H, | (M − 2) |
| | s), 7.13 (1H, d), 6.89 (1H, d), 6.84 (1H, d), 6.78 (1H, d), | |
| | 5.97 (1H, d), 5.49 (1H, d), 3.85 (3H, s), 3.01 (2H, q), | |
| | 1.94 (1H, m), 1.87 (1H, m), 1.63 (2H, m), 1.37 (6H, d), (NH₂ | |
| | proton exchanged due to moisture) | |
| 70 | δ 9.45 (1H, s), 8.19 (1H, s), 7.60 (1H, t), 7.54 (1H, d), | 709.31 |
| | 7.10-7.18 (5H, m), 6.82 (1H, d), 6.77 (1H, d), 6.03 (1H, | (M + 1) |
| | d), 5.35 (1H, d), 3.61 (4H, t), 3.55 (2H, s), 2.94 (2H, s), | |
| | 2.59 (4H, s), 2.29 (3H, s), 1.82 (2H, m), 1.74-1.78 (2H, | |
| | m), 1.35 (6H, d), (NH₂ proton exchanged due to | |
| | moisture) | |
| 71 | δ 9.47 (1H, s), 8.18 (1H, s), 7.79 (1H, t), 7.47 (1H, d), | 600.20 |
| | 7.36 (1H, d), 7.05 (2H, s), 6.84 (1H, d), 6.79 (1H, d), | (M + 1) |
| | 6.73 (1H, s), 6.18 (2H, s), 5.97 (1H, s), 5.91 (1H, s), | |
| | 5.28 (2H, s), 3.23 (3H, s), 3.05 (2H, s), 1.39 (6H, s), (NH₂ | |
| | proton exchanged due to moisture) | |
| 72 | δ 9.45 (1H, s), 8.20 (1H, s), 7.61 (1H, t), 7.50-7.54 (2H, | 701.25 |
| | m), 7.26 (1H, t), 7.21 (1H, d), 6.93 (1H, d), 6.84 (1H, d), | (M + 1) |
| | 6.78 (1H, d), 5.97 (1H, d), 5.50 (1H, d), 3.60 (4H, t), | |
| | 3.54 (2H, s), 2.99 (2H, q), 2.56 (4H, m), 1.94-1.99 (1H, m), | |
| | 1.81-1.87 (1H, m), 1.68-1.74 (1H, m), 1.52-1.58 (1H, m), | |
| | 1.36 (6H, d), (NH₂ proton exchanged due to moisture) | |
| 73 | δ 9.45 (1H, s), 8.17 (1H, s), 7.71 (1H, t), 7.55 (1H, d), | 627.20 |
| | 7.37 (1H, d), 7.07 (2H, t), 6.83 (1H, d), 6.77 (1H, d), | (M + 1) |
| | 6.21 (2H, t), 5.23 (2H, s), 3.01 (2H, s), 2.45 (2H, d), | |
| | 2.04-2.10 (1H, m), 1.74-1.77 (2H, m), 1.57-1.59 (2H, m), | |
| | 1.48-1.55 (2H, m), 1.36 (6H, s), 1.29-1.34 (2H, m), (NH₂ proton | |
| | exchanged due to moisture) | |
| 74 | δ 9.47 (1H, s), 9.36 (1H, s), 8.18 (1H, s), 7.78 (1H, t), | 613.12 |
| | 7.46 (1H, d), 7.28 (1H, d), 7.11 (1H, t), 7.00 (2H, s), | (M + 1) |
| | 6.82 (2H, dd), 6.64-6.71 (3H, m), 6.15 (2H, s), 5.27 (2H, s), | |
| | 3.05 (2H, s), 1.39 (6H, s), (NH₂ proton exchanged due to | |
| | moisture) | |
| 75 | δ 9.45 (1H, s), 8.21 (1H, s), 7.66 (1H, t), 7.48 (1H, t), | 657.18 |
| | 7.25-7.28 (2H, m), 7.18 (1H, s), 6.84-6.87 (2H, m), | (M + 1) |
| | 6.74-6.79 (2H, m), 5.96-6.01 (2H, m), 5.90 (1H, d), 5.52 (1H, | |
| | d), 3.21 (3H, s), 3.00 (2H, q), 1.98 (1H, m), 1.82-1.83 (1H, | |
| | m), 1.64 (1H, m), 1.40 (1H, m), 1.38 (3H, s), 1.36 (3H, s), | |
| | (NH₂ proton exchanged due to moisture) | |
| 76 | δ 9.44 (1H, s), 8.20 (1H, s), 7.50-7.57 (2H, m), 7.42 (1H, | 642.19 |
| | d), 7.23 (1H, s), 7.13 (1H, d), 6.91 (1H, d), 6.83 (1H, d), | (M + 1) |
| | 6.77 (1H, d), 5.95 (1H, d), 5.47 (1H, d), 2.95 (2H, q), | |
| | 1.97 (1H, m), 1.80-183 (1H, m), 1.72-1.76 (1H, m), | |
| | 1.55-1.61 (2H, m), 1.37 (3H, s), 1.36 (3H, s), 0.89-0.92 (2H, m), | |
| | 0.76-0.79 (2H, m), (NH₂ proton exchanged due to | |
| | moisture) | |
| 77 | δ 9.48 (1H, s), 8.20 (1H, s), 7.80-7.86 (1H, m), 7.44 (2H, | 628.11 |
| | dd), 6.76-6.84 (3H, m), 5.92-5.97 (4H, m), 5.16 (2H, d), | (M + 1) |
| | 3.23 (3H, s), 3.05 (2H, m), 2.06 (3H, s), 2.02 (3H, s), | |
| | 1.39 (6H, s), (NH₂ proton exchanged due to moisture) | |
| 78 | δ 8.20 (1H, s), 7.68 (1H, t), 7.38 (1H, d), 7.27 (1H, d), | 577.14 |
| | 6.98 (2H, q), 5.96 (2H, s), 5.15 (2H, s), 3.81 (3H, s), | (M + 1) |
| | 3.05 (2H, s), 2.75 (3H, s), 2.06 (6H, s), 1.38 (6H, s), (NH₂ | |
| | proton exchanged due to moisture) | |
| 80 | δ 9.47 (1H, s), 8.19 (1H, s), 7.76 (1H, t), 7.66 (1H, d), | 672.23 |
| | 7.41 (1H, d), 6.80 (2H, dd), 5.97 (2H, s), 5.12 (2H, s), | (M + 1) |
| | 3.60 (4H, t), 3.54 (2H, s), 3.02 (2H, s), 2.50 (4H, m), 2.06 (6H, | |
| | s), 1.38 (6H, s), (NH₂ proton exchanged due to moisture) | |
| 81 | δ 9.54 (1H, s), 8.18 (1H, s), 7.51 (1H, t), 7.24 (1H, d), | 570.6 |
| | 6.99-7.18 (5H, m), 7.00 (1H, d), 6.74 (1H, d), 6.02 (1H, | (M + 1) |
| | d), 5.30 (1H, d), 2.80 (4H, m), 2.76 (3H, s), 2.27 (5H, m), | |
| | 1.90-1.93 (2H, m), 1.75 (2H, m), (NH₂ proton exchanged | |
| | due to moisture) | |
| 82 | δ 9.45 (1H, s), 8.18 (1H, s), 7.73 (1H, s), 7.56-7.64 (2H, | 708.30 |
| | m), 7.21-7.31 (4H, m), 7.05 (2H, d), 6.76-6.83 (2H, m), | (M + 1) |
| | 5.90 (1H, d), 5.37 (1H, d), 3.63 (2H, s), 3.16 (2H, s), | |
| | 3.12 (2H, s), 2.95 (2H, q), 2.73 (2H, m), 2.00 (2H, m), | |
| | 1.70-1.72 (2H, m), 1.36 (6H, s), (NH₂ proton exchanged due | |
| | to moisture) | |
| 83 | δ 9.45 (1H, s), 8.19 (1H, s), 7.65 (1H, m), 7.54 (1H, m), | 711.20 |
| | 7.21-7.30 (4H, m), 7.05 (2H, d), 6.79 (2H, q), 5.90 (1H, | (M+) |
| | d), 5.37 (1H, d), 3.54 (2H, s), 2.99 (2H, q), 2.80 (4H, t), | |
| | 2.62 (4H, t), 1.97-2..04 (2H, m), 1.65-1.74 (2H, m), | |
| | 1.36 (6H, s), (NH₂ proton exchanged due to moisture) | |
| 84 | δ 8.18 (1H, s), 7.66 (1H, t), 7.35 (1H, d), 7.28 (1H, d), | 549.11 |
| | 7.04 (2H, m), 6.97 (2H, m), 6.20 (2H, t), 5.26 (2H, s), | (M + 1) |
| | 3.80 (3H, s), 3.04 (2H, s), 2.73 (3H, s), 1.36 (6H, s), (NH₂ | |
| | proton exchanged due to moisture) | |
| 85 | δ 9.46 (1H, s), 8.19 (1H, s), 7.91 (1H, s), 7.76 (1H, t), | 643.30 |
| | 7.55 (1H, s), 7.46 (1H, d), 7.34 (1H, d), 6.84 (1H, d), | (M + 1) |
| | 6.79 (1H, d), 5.93 (2H, s), 5.15 (2H, s), 4.13 (2H, q), 3.04 (2H, | |
| | s), 2.05 (6H, s), 1.36-1.40 (9H, m), (NH₂ proton | |
| | exchanged due to moisture) | |
| 86 | δ 12.84 (1H, s), 9.47 (1H, s), 8.20 (1H, s), 7.89 (1H, s), | 615.18 |
| | 7.77 (1H, t), 7.61 (1H, s), 7.47 (1H, d), 7.35 (1H, d), | (M + 1) |
| | 6.85 (1H, d), 6.79 (1H, d), 5.93 (2H, s), 5.16 (2H, s), 3.05 (2H, | |
| | s), 2.05 (6H, s), 1.39 (6H, s), (NH₂ proton exchanged due | |
| | to moisture) | |
| 87 | δ 9.47 (1H, s), 8.19 (1H, s), 7.85 (1H, d), 7.66 (1H, t), | 629.02 |
| | 7.44 (1H, d), 6.81 (2H, dd), 5.97 (2H, s), 5.15 (2H, s), | (M + 2), |
| | 3.02 (2H, s), 2.06 (6H, s), 1.38 (6H, s), (NH₂ protons | 627.08 |
| | exchanged due to moisture) | (M+) |
| 88 | δ 9.47 (1H, s), 8.20 (2H, s), 7.88 (1H, t), 7.62 (1H, t), | 549.10 |
| | 7.50 (1H, d), 6.83 (2H, dd), 5.98 (2H, s), 5.16 (2H, s), | (M + 1) |
| | 3.04 (2H, s), 2.07 (6H, s), 1.38 (6H, s), (NH₂ protons | |
| | exchanged due to moisture) | |
| 89 | δ 9.46 (1H, s), 8.19 (1H, s), 7.90 (1H, s), 7.76 (1H, t), | 657.27 |
| | 7.56 (1H, s), 7.46 (1H, d), 7.34 (1H, t), 6.81 (2H, dd), | (M + 1) |
| | 5.93 (2H, s), 5.14 (2H, s), 4.05 (2H, t), 3.04 (2H, s), | |
| | 2.05 (6H, s), 1.74-1.83 (2H, m), 1.38 (6H, s), 0.85 (3H, t), | |
| | (NH₂ protons exchanged due to moisture) | |
| 90 | δ 9.44 (1H, s), 8.19 (1H, s), 7.89 (1H, s), 7.63 (1H, t), | 666.28 |
| | 7.54 (1H, s), 7.34 (1H, d), 7.27 (2H, t), 7.17 (2H, dd), | (M + 1) |
| | 7.00 (2H, d), 6.82 (2H, dd), 5.91 (1H, d), 5.36 (1H, d), | |
| | 4.12 (2H, q), 2.98 (2H, q), 1.98-2.01 (2H, m), | |
| | 1.67-1.72 (2H, m), 1.36-1.40 (9H, m), (NH₂ protons exchanged due | |
| | to moisture) | |
| 91 | δ 9.49 (1H, s), 8.20 (1H, s), 7.80 (1H, t), 7.50 (1H, d), | 631.14 |
| | 7.43-7.46 (3H, m), 7.09 (1H, d), 6.86 (1H, d), 6.80 (1H, | (M + 1) |
| | d), 5.91 (2H, s), 5.18 (2H, s), 3.06 (2H, s), 2.04 (6H, s), | |
| | 1.39 (6H, s), (NH₂ protons exchanged due to moisture) | |
| 92 | δ 9.45 (1H, s), 8.07 (1H, s), 7.78 (1H, t), 7.62 (1H, d), | 549.18 |
| | 7.42 (1H, d), 7.14 (1H, s), 6.82 (1H, d), 6.75 (1H, d), | (M + 1) |
| | 6.10 (1H, s), 5.76-5.80 (1H, m), 5.74 (2H, s), 2.87 (2H, q), | |
| | 2.73 (3H, s), 1.79 (3H, d), 1.31 (3H, s), 1.14 (3H, s), (NH₂ | |
| | protons exchanged due to moisture) | |
| 93 | δ 9.59 (1H, s), 8.19 (1H, s), 7.86 (1H, s), 7.76 (1H, t), | 599.33 |
| | 7.55 (1H, s), 7.44 (1H, d), 7.33 (1H, d), 7.12 (1H, d), | (M + 1) |
| | 6.76 (1H, d), 5.93 (2H, s), 5.16 (2H, s), 3.84 (3H, s), 2.88 (2H, | |
| | t), 2.82 (2H, t), 2.05 (6H, s), 1.94-2.00 (2H, m), (NH₂ | |
| | protons exchanged due to moisture) | |
| 94 | δ 9.59 (1H, s), 8.19 (1H, s), 7.91 (1H, s), 7.76 (1H, t), | 613.25 |
| | 7.56 (1H, s), 7.45 (1H, d), 7.32 (1H, d), 7.12 (1H, d), | (M + 1) |
| | 6.77 (1H, d), 5.93 (2H, s), 5.15 (2H, s), 4.13 (2H, q), 2.88 (2H, | |
| | t), 2.82 (2H, t), 2.05 (6H, s), 1.96 (2H, t), 1.40 (3H, t), | |
| | (NH₂ protons exchanged due to moisture) | |
| 95 | δ 9.44 (1H, s), 8.07 (1H, s), 7.80 (1H, t), 7.63 (1H, d), | 577.22 |
| | 7.44 (1H, d), 6.76 (2H, q), 5.89 (1H, d), 5.78 (1H, q), | (M + 1) |
| | 5.58 (1H, d), 2.84 (2H, q), 2.72 (3H, s), 2.01 (3H, s), 1.78 (3H, | |
| | d), 1.29 (3H, s), 1.22 (6H, d), (NH₂ protons exchanged | |
| | due to moisture) | |
| 96 | δ 9.44 (1H, s), 8.06 (1H, s), 7.77 (1H, t), 7.62 (1H, d), | 549.31 |
| | 7.41 (1H, d), 7.14 (1H, s), 6.82 (1H, d), 6.75 (1H, d), | (M + 1) |
| | 6.09 (1H, s), 5.79 (1H, q), 5.74 (2H, s), 2.87 (2H, q), 2.73 (3H, | |
| | s), 1.79 (3H, d), 1.31 (3H, s), 1.14 (3H, s), (NH₂ protons | |
| | exchanged due to moisture) | |
| 99 | δ 9.44 (1H, s), 8.07 (1H, s), 7.83-7.89 (2H, m), 7.68 (1H, | 629.06 |
| | d), 7.53 (1H, s), 7.49 (1H, d), 7.13 (1H, s), 6.82 (1H, d), | (M + 1) |
| | 6.75 (1H, d), 6.07 (1H, s), 5.80-5.84 (1H, m), 5.78 (1H, | |
| | s), 5.71 (1H, s), 4.11 (2H, q), 2.87 (2H, q), 1.80 (3H, d), | |
| | 1.36 (3H, t), 1.29 (3H, s), 1.17 (3H, s), (NH₂ protons | |
| | exchanged due to moisture) | |
| 100 | δ 9.21 (1H, s), 8.47 (1H, s), 7.69 (1H, t), 7.35 (2H, dd), | 534.97 |
| | 7.04 (2H, t), 6.83 (2H, dd), 6.20 (2H, t), 5.38 (2H, s), | (M+) |
| | 3.02 (2H, s), 2.73 (3H, s), 2.44 (3H, s), 1.39 (6H, s), (NH₂ | |
| | protons exchanged due to moisture) | |

### Pharmaceutical Compositions

In another embodiment present invention provides a pharmaceutical composition comprising a therapeutically effective amount of one or more of a compound of formula (I) or (Ia). While it is possible to administer therapeutically effective quantity of compounds of formula (I) or (Ia) either individually or in combination, directly without any formulation, it is common practice to administer the compounds in the form of pharmaceutical dosage forms comprising pharmaceutically acceptable excipient(s)/adjuvant(s) or carrier and at least one active ingredient. These dosage forms may be administered by a variety of routes including oral, topical, transdermal, subcutaneous, intramuscular, intravenous, intraperitoneal, intranasal, pulmonary etc.

Oral compositions may be in the form of solid or liquid dosage form. Solid dosage form may comprise pellets, pouches, sachets or discrete units such as tablets, multi-particulate units, capsules (soft & hard gelatin) etc. Liquid dosage forms may be in the form of elixirs, suspensions, emulsions, solutions, syrups etc. Composition intended for oral use may be prepared according to any method known in the art for the manufacture of the composition and such pharmaceutical compositions may contain in addition to active ingredients, excipients such as diluents, disintegrating agents, binders, solubilizers, lubricants, glidants, surfactants, suspending agents, emulsifiers, chelating agents, stabilizers, flavours, sweeteners, colours etc. Some example of suitable excipients include lactose, cellulose and its derivatives such as microcrystalline cellulose, methylcellulose, hydroxy propyl methyl cellulose & ethylcellylose, dicalcium phosphate, mannitol, starch, gelatin, polyvinyl pyrolidone, various gums like acacia, tragacanth, xanthan, alginates & its derivatives, sorbitol, dextrose, xylitol, magnesium stearate, talc, colloidal silicon dioxide, mineral oil, glyceryl mono stearate, glyceryl behenate, sodium starch glycolate, cross povidone, crosslinked carboxymethylcellulose, various emulsifiers such as polyethylene glycol, sorbitol, fatty acid esters, polyethylene glycol alkylethers, sugar esters, polyoxyethylene polyoxypropyl block copolymers, polyethoxylated fatty acid monoesters, diesters and mixtures thereof.

Intranasal or pulmonary compositions according to present invention can be in the form of liquid or solid or semisolid composition suitable for nasal administration. Liquid composition can be aqueous, non-aqueous composition, suspension or emulsion, solid composition can be in the form of powder and the like and semi solid composition can be in form of gel and the like. Nasal/pulmonary compositions may also form in-situ gel. Said nasal or pulmonary composition comprises compounds of formula (I) or (Ia) optionally with one or more suitable excipients selected from in-situ gelling agent, mucoadhesive agent, polymer, humectant, buffering agent, stabilizer, surfactant, preservative, thickening agent, solvents, co-solvents, permeation enhancer, chelating agent, viscosity modifying agent, sweetener, taste masking agent, solubilizer, flavoring agent, emulsifier and isotonicity agent.

Sterile compositions for injection can be formulated according to conventional pharmaceutical practice by dissolving or suspending the active substance in a vehicle such as water for injection, N-Methyl-2-Pyrrolidone, propylene glycol and other glycols, alcohols, a naturally occurring vegetable oil like sesame oil, coconut oil, peanut oil, cotton seed oil or a synthetic fatty vehicle like ethyl oleate or the like. Buffers, anti-oxidants, preservatives, complexing agents like cellulose derivatives, peptides, polypeptides and cyclodextrins and the like can be incorporated as required.

The dosage form can have a slow, delayed or controlled release of active ingredients in addition to immediate release dosage forms.

The amount of active ingredient which is required to achieve a therapeutic effect will, of course, vary with the particular compound, the route of administration, the subject under treatment, and the particular disorder or disease being treated. The compounds of the invention may be administered by oral, inhalation or parenteral route at a dose of from 0.0005 to 100 mg/kg per day, preferably from 0.0005 to 50 mg/kg per day, more preferably from 0.0001 to 20 mg/kg per day, most preferably from 0.0001 to 10 mg/kg per day. The dose range for adult humans is generally from 5 μg to 5 g per day, preferably dose range is 10 μg to 2 g per day.

Dosage forms of presentation provided in discrete units may conveniently contain an amount of compound of the invention which is effective at such dosage or as a multiple of the same, for example units containing 5 μg to 1000 mg.

In another embodiment present invention provides method of treating allergic or non-allergic airway disease by administering a therapeutically effective amount of a compound of formula (I) or (Ia) to a mammal, including human being, in need thereof. Allergic and non-allergic airway diseases include allergic and non-allergic asthma, chronic obstructive pulmonary disease (COPD), rhinitis, chronic bronchitis, emphysema, or asthma-like syndrome such as coughing, wheezing or dyspnea.

A preferred embodiment of the present invention is a method for treating chronic obstructive pulmonary disease or asthma by administering a therapeutically effective amount of a compound of formula (I) or (Ia) to a mammal, including human being, in need thereof.

A most preferred embodiment of the present invention is a method for treating chronic obstructive pulmonary disease by administering a therapeutically effective amount of a compound of formula (I) or (Ia) to a mammal, including human being, in need thereof.

Another embodiment of the present invention is the use of a compound of formula (I) or (Ia) for the preparation of a medicament for treating allergic or non-allergic airway disease in a mammal, including human being.

A preferred embodiment of the present invention is the use of a compound of formula (I) or (Ia) for the preparation of a medicament for treating chronic obstructive pulmonary disease or asthma in a mammal, including human being.

A most preferred embodiment of the present invention is the use of a compound of formula (I) or (Ia) for the preparation of a medicament for treating chronic obstructive pulmonary disease.

### Biological Testing

### Biological Example 1

### In-Vitro Studies

### Inhibition of PI3-Kinase Enzymatic Activity (HTRF Enzyme Assay)

Inhibition of PI3-Kinase enzymatic activity was determined using a homogeneous time-resolved fluorescence (HTRF) kit from Eurofins. 1 μM of Compounds of present invention were premixed with DMSO. The experiment was initiated by mixing 1.0% DMSO as vehicle/compounds with purified recombinant human PI3K enzyme {δ (50 ng/well); γ (100 ng/well)} (Invitrogen, USA) and substrate (PIP2, 10 μM) in the wells and incubation for 15 min at room temperature. There after ATP was added (δ at 200 μM, and γ at 50 μM) in to the wells containing reaction mixture, followed by re-incubation for 30 minutes at room temperature. Reaction was terminated by addition of a stopping solution and was further incubated for 4 hours at room temperature before reading using Envision multimode reader (Perkin Elmer). Percentage inhibition of PI3K activity was calculated by determining ratio of specific europium 665 nm energy transfer signal to reference 615 nm signals. Results are summarized in the table given below.

TABLE 2

| Compound | | |
|---|---|---|
| No | δ | γ |
| 1 | ++++ | ++++ |
| 2 | ++++ | ++++ |
| 3 | ++++ | ++++ |
| 4 | ++++ | ++ |
| 5 | ++++ | ++++ |
| 6 | ++++ | +++ |
| 7 | ++++ | +++ |
| 8 | ++++ | +++ |
| 9 | ++++ | ++++ |
| 10 | ++++ | +++ |
| 11 | ++++ | +++ |
| 12 | ++++ | ++++ |
| 13 | ++++ | ++++ |
| 14 | ++++ | ++++ |
| 15 | ++++ | ++++ |
| 16 | ++++ | +++ |
| 17 | +++ | +++ |
| 18 | ++++ | +++ |
| 19 | ++++ | ++++ |
| 20 | ++++ | ++++ |
| 21 | ++++ | +++ |
| 22 | ++++ | +++ |
| 23 | ++++ | ++++ |
| 24 | ++++ | ++++ |
| 25 | ++++ | ++++ |
| 26 | ++++ | ++ |
| 28 | ++++ | ++++ |
| 29 | ++++ | ++++ |
| 30 | ++++ | +++ |
| 31 | ++++ | +++ |
| 33 | ++++ | +++ |
| 35 | ++++ | +++ |
| 37 | ++++ | ++++ |
| 39 | ++++ | ++++ |
| 40 | ++++ | +++ |
| 41 | ++++ | ++++ |
| 42 | ++++ | ++++ |
| 43 | ++++ | ++++ |
| 44 | ++++ | ++++ |
| 45 | ++++ | +++ |
| 48 | ++++ | ++++ |
| 55 | ++++ | +++ |
| 56 | ++++ | +++ |
| 57 | ++++ | +++ |
| 58 | ++++ | ++++ |
| 59 | ++++ | +++ |
| 60 | ++++ | +++ |
| 62 | ++++ | +++ |
| 67 | ++++ | − |
| 68 | ++++ | ++++ |
| 69 | ++++ | +++ |
| 71 | ++++ | ++++ |
| 72 | ++++ | ++++ |
| 75 | ++++ | ++++ |
| 76 | ++++ | ++++ |
| 77 | ++++ | ++++ |
| 78 | ++++ | +++ |
| 80 | ++++ | +++ |
| 84 | ++++ | +++ |
| 85 | ++++ | ++++ |
| 88 | ++++ | ++++ |
| 89 | ++++ | ++++ |
| 93 | ++++ | ++++ |
| 94 | ++++ | ++++ |
| 95 | ++++ | ++++ |
| 96 | ++++ | ++++ |
| 99 | ++++ | ++ |

Criteria:
++++ = Inhibition ≥80% ≤100%;
+++ = Inhibition ≥60% <80%;
++ = Inhibition ≥40% <60%;
+ = Inhibition ≥20% <40%;
− = Inhibition <20%;
NA—Not Available

Observation: in-vitro data shows that compounds of present invention effectively inhibits PI3K activity.

### Biological Example 2

### In Vivo Studies

### In Vivo Efficacy Evaluation of Compounds in Animal Model of Airway Inflammation (COPD):

The tobacco smoke induced airway inflammation model is used for in vivo efficacy of compound. Many investigators have used acute tobacco smoke (TS) exposure in rodents as models of airway inflammation for quick screening of anti-inflammatory therapies for COPD (J Pharmacol Exp Ther. 2008; 324(3):921-9; J Pharmacol Exp Ther. 2010; 332(3):764-75; Journal of Inflammation 2013, 10(Suppl 1):31 and Eur Respir J Suppl 2006; 663s:3850). Given its position as predominant cause of COPD, animal models using TS exposure would appear to be the logical choice for investigation (Respir Res. 2004; 2;5:18).

### Efficacy Studies in Acute Guinea Pig Model of Airway Inflammation

Guinea pigs were exposed to tobacco smoke (TS) in an acrylic chamber. Animals were exposed to TS from 5, 10, 15 cigarettes on day 1, day 2, day 3 respectively. From day 4 onwards till day 11, animals were exposed to TS from 15 cigarettes per day. On 11 days of exposure of guinea pig to TS, significant inflammatory cell recruitment, predominantly neutrophils, to lungs was observed as compared to air exposed control guinea pig (BALF neutrophil levels, 0.59±0.15*10⁶ cells/animal in air control group vs 8.3±1.4*10⁶cells/animal in smoke exposed vehicle group)

Lung delivery of test compound was achieved by whole body aerosol exposure using nebulizer for 56 minutes in a chamber. Guinea pig were divided in different dose groups and exposed in a chamber for 56 minutes with vehicle or Compound No. 39 (1 mg/ml or 3 mg/ml). A total quantity of 6.0 ml of either vehicle or test compound formulation was nebulized in chambers to respective groups over 56 mins period. Test compound was administered 2 hr prior to TS exposure from day 6 to day 11. Bronchoalveolar lavage (BAL) was performed 24 hr post last TS exposure.

Trachea of animal was cannulated using catheter. Phosphate Buffer Saline (PBS) was used as lavage fluid. A volume of 5.0 ml was gently instilled and withdrawn and collected in microcentrifuge tube placed on ice. This procedure was repeated further 5 times.

Lavage fluid was separated from cells by centrifugation and supernatant separated. The cell pallet was resuspended in known volume of PBS. Cells in aliquot were stained using Turk solution and total cell numbers were calculated by counting Turk stained aliquot under microscope using haemocytometer.

The residual cell suspension was resuspended and slides prepared using cyto centrifuge technique (Cytospin 4, Thermo Shandon). The slides were then fixed with methanol, air dried and stained with May Grunwald Giemsa stain. Up to 300 cells were counted and differentiated using standard morphometric techniques under light microscopy.

All results are presented at individual data for each animal and mean value calculated for each group. Percentage inhibition for the neutrophil was calculated for Compound No 39 treatment group against vehicle group. Results are summarized herein below:

The effect of treatment Compound No 39 on cigarette smoke induced Neutrophil accumulation in BAL Fluid.

TABLE 3

| | | Exposure | Neutrophil | |
|---|---|---|---|---|
| | | Duration | (*10⁶ cells/ | |
| Treatment | Concentration | (Minutes) | animal) | % Inhibition |
| Vehicle | NA | 56 | 8.3 ± 1.4 | |
| Compound | 1 mg/ml | 56 | 6.5 ± 3.2 | 22 |
| No. 39 | 3 mg/ml | 56 | 2.7 ± 1.4 | 68 |

Values are Mean ± SEM;
NA: Not applicable

Observation: It was observed that compounds of present invention were found effective in inhibition of neutrophil, an index of pulmonary inflammation in guinea pig model of airway inflammation. These results indicate that compounds of present invention possess pulmonary anti-inflammatory activity.

### In Vivo Efficacy Evaluation of Compounds in Animal Model of Airway Inflammation (Asthma):

Allergen chicken egg ovalbumin (OVA) model, which shares features similar to human allergic asthma, including the presence of eosinophilic and lymphocytic lung inflammation, is frequently used for model for asthma. Ovalbumin is an inert protein that is not intrinsically immunogenic and therefore needs to be injected systemically in the presence of an adjuvant, typically aluminum hydroxide (alum), to induce Th2-driven response in mice (Curr. Protoc. Mouse Biol. 6:169-184, 2016).

Ovalbumin (20 μg) (OVA, Sigma Aldrich, St. Louis, Mo.), emulsified with inject alum (1 mg, 0.2 mL) was administered in mice intraperitoneally (i.p.) on days 1 and 14 for sensitization, and then challenged with OVA (0.5% w/v) exposure for 30 minutess using nebulizer on days 21, 22 and 23 in Compound no 44 treated and vehicle control animals. Saline control mice received normal saline as sensitization and challenge dose in similar fashion.

Lung delivery of compound of present invention was achieved by whole body aerosol exposure using nebulizer for 25 minutes in a chamber. Mice were divided in different dose groups and exposed in a chamber for 25 minutes with vehicle or Compound no 44 (1 mg/ml or 3 mg/ml). Test compound/vehicle was administered once daily for 5 days starting from day 20 till day 24. On each challenge day treatment was administered 2 hr before OVA challenge. Bronchoalveolar lavage (BAL) was performed 48 hr post last OVA exposure (i.e. on day 25).

Trachea of animal was cannulated using catheter. Phosphate Buffer Saline (PBS) was used as lavage fluid. A volume of 0.5 ml was gently instilled and withdrawn and collected in microcentrifuge tube placed on ice. This procedure was repeated further 3 times.

Lavage fluid was separated from cells by centrifugation and supernatant separated. The cell pallet was resuspended in known volume of PBS. Cells in aliquot were stained using Turk solution and total cell numbers were calculated by counting Turk stained aliquot under microscope using haemocytometer.

The residual cell suspension was resuspended with PBS and slides prepared using cyto centrifuge technique (Cytospin 4, Thermo Shandon). The slides were then fixed with methanol, air dried and stained with May Grunwald Giemsa stain. Up to 300 cells were counted and differentiated using standard morphometric techniques under light microscopy.

All results are presented at individual data for each animal and mean value calculated for each group. Percentage inhibition for each cell type is calculated as below.

% inhibition=[(Vehicle treatment−Drug treatment)/(Vehicle treatment−Saline treatment)]*100

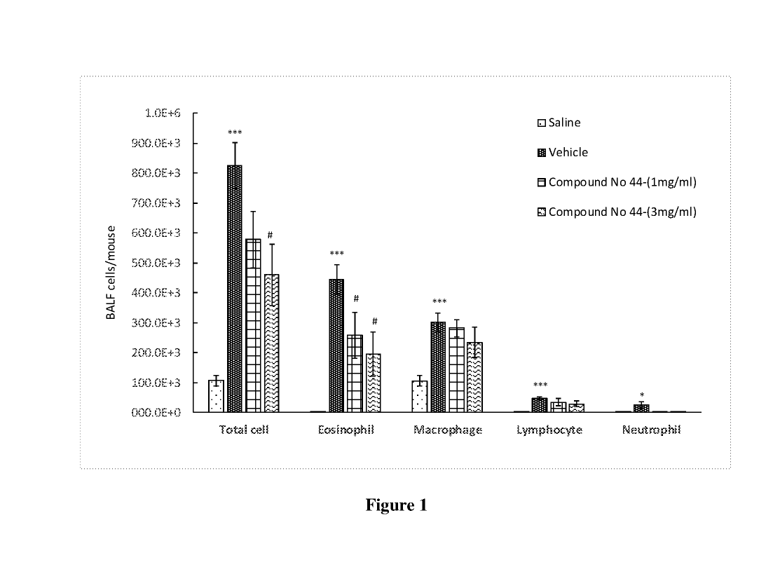

The effect of treatment with Compound no 44 on OVA induced pulmonary inflammatory cell accumulation in BAL fluid is shown in FIG. 1.

### Observation

Compound of present invention effectively reduced OVA-induced lung inflammatory response in a dose dependent manner; significantly reduced eosinophil influx (42% and 56%), lymphocyte influx (27% and 39%) at doses 1 mg/ml and 3 mg/ml respectively.

1. A compound of formula (I) 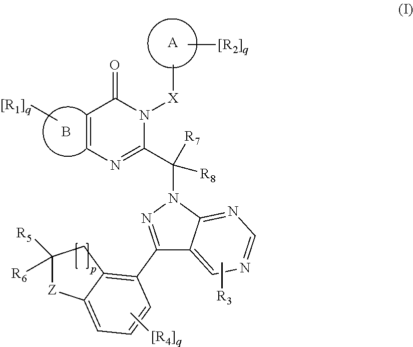
wherein
X is a bond or 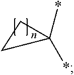
* denotes point of attachment to ring A and to the rest of molecule;
when X is a bond, ring A is a mono or bicyclic heteroaryl containing at least one N and said N is point of attachment to X;
or when X is 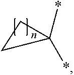
ring A is mono or bicyclic aryl or heteroaryl;
ring B is mono or bicyclic aryl or 6-membered heteroaryl;
R₁ and R₂ are independently selected from hydrogen, halogen, NO₂, NR₁₁R₁₂, CF₃, CN, COOR₉, COR₉, OR₉, OCOR₉, O—(C₁-C₆)alkyl-OR₉, O—(C₁-C₆)alkyl-S(O)ₜR₉, O—(C₁-C₆)alkyl-NR₁₁R₁₂, O—(C₁-C₆)alkyl-COOR₉, O—(C₁-C₆)alkyl-COR₉, S(O)ₜR₉, (C₁-C₆)alkyl, (C₃-C₆)cycloalkyl, (C₁-C₆)alkyl-OR₉, (C₁-C₆)alkyl-S(O)ₜR₉, (C₁-C₆)alkyl-NR₁₁R₁₂, (C₁-C₆)alkylaryl, (C₁-C₆)alkylheteroaryl, (C₁-C₆)alkylheterocycloalkyl, (C₁-C₆)alkylcycloalkyl, (C₂-C₅)alkenyl-R₁₃, (C₂-C₅)alkynyl-R₁₃, heterocycloalkyl, aryl, boronate ester and heteroaryl; said (C₃-C₆)cycloalkyl, heterocycloalkyl, aryl and heteroaryl are optionally substituted by R₁₀;
R₃ and R₄ are independently selected from hydrogen, OR₉, halogen, NR₁₁R₁₂, NO₂, CF₃, O—(C₁-C₆)alkyl-OR₉, O—(C₁-C₆)alkyl-S(O)ₜR₉O—(C₁-C₆)alkyl-NR₁₁R₁₂, O—(C₁-C₆)alkyl-COOR₉O—(C₁-C₆)alkyl-COR₉, S(O)ₜR₉, COR₉, COOR₉, (C₁-C₆)alkyl, (C₃-C₆)cycloalkyl, (C₂-C₅)alkenyl-R₁₃, (C₂-C₅)alkynyl-R₁₃ aryl, heteroaryl and heterocycloalkyl; the said (C₃-C₆)cycloalkyl, heterocycloalkyl, aryl and heteroaryl are optionally substituted by R₁₀;
R₅, R₆, R₇ and R₈ are independently selected from hydrogen, halogen, NR₁₁R₁₂, CF₃, COOR₉, COR₉, and (C₁-C₆)alkyl, or
R₅ and R₆ or R₇ and R₈ together may form 3 to 6 membered monocyclic cycloalkyl ring;
R₉ is independently selected from hydrogen, NR₁₁R₁₂, CF₃, SO₃H, glucuronate, (C₁-C₆)alkyl, (C₁-C₆)alkyl-R₁₀, (C₃-C₆)cycloalkyl, heterocycloalkyl, aryl and heteroaryl; said (C₃-C₆)cycloalkyl, heterocycloalkyl, aryl and heteroaryl are optionally substituted by R₁₀;
R₁₀ is independently selected from hydrogen, oxo, halogen, CF₃, S(O)ₜR₉, OR₉, NO₂, COR₉, COOR₉, NR₁₁R₁₂, N(R₉)COR₉, N(R₉)S(O)ₘR₉, OCOR₉, (C₁-C₆)alkyl, (C₃-C₆)cycloalkyl, (C₁-C₆)alkyl-OR₉, (C₁-C₆)alkyl-COOR₉, (C₁-C₆)alkyl-COR₉, (C₁-C₆)alkyl-S(O)ₜR₉, (C₁-C₆)alkyl-NHCOR₉, (C₁-C₆)alkyl-NHS(O)ₜR₉, (C₁-C₆)alkyl-NR₁₁R₁₂, heterocycloalkyl, aryl, heteroaryl, (C₂-C₅)alkenyl-R₁₃ and (C₂-C₅)alkynyl-R₁₃;
R₁₁ and R₁₂ are independently selected from hydrogen, COR₉, N(R₉)₂, N(R₉)S(O)ₜR₉, N(R₉)COR₉, CF₃, S(O)ₜR₉, (C₁-C₆)alkyl, fluoro(C₁-C₆)alkyl, aryl, heteroaryl, heterocycloalkyl, (C₃-C₆)cycloalkyl, (C₁-C₆)alkyl(C₃-C₆)cycloalkyl, (C₁-C₆)alkylheterocycloalkyl, (C₁-C₆)alkylaryl, (C₁-C₆)alkylheteroaryl, (C₁-C₆)alkyl-OR₉, (C₁-C₆)alkyl-S(O)ₜR₉, (C₁-C₆)alkyl-COOR₉, (C₁-C₆)alkyl-COR₉, (C₁-C₆)alkyl-OCOOR₉, (C₁-C₆)alkyl-N(R₉)COR₉ and (C₁-C₆)alkyl-N(R₉)S(O)ₘR₉; or
R₁₁ and R₁₂ together with N, may form a 3 to 8 member monocyclic or 8 to 12 membered bicyclic heterocycle ring, wherein the said mono and bicyclic ring may additionally contain 1, 2 and 3 ring heteroatoms selected from O, S(O)ₜ or N; said heterocycle is optionally substituted by R₁₀;
R₁₃ is independently selected from hydrogen, (C₁-C₆)alkyl-OR₉, (C₁-C₆)alkyl-S(O)ₜR₉, (C₁-C₆)alkyl-COOR₉, (C₁-C₆)alkyl-COR₉, (C₁-C₆)alkyl-OCOOR₉, (C₁-C₆)alkyl-N(R₉)COR₉, (C₁-C₆)alkyl-N(R₉)S(O)ₘR₉, (C₁-C₆)alkyl-NR₁₁R₁₂, aryl, heteroaryl, (C₃-C₆)cycloalkyl, heterocycloalkyl, (C₁-C₆)alkyl(C₃-C₆)cycloalkyl, (C₁-C₆)alkylheterocycloalkyl, (C₁-C₆)alkylheteroaryl and (C₁-C₆)alkylaryl; said C₁-C₆)alkyl(C₃-C₆)cycloalkyl, (C₁-C₆)alkylheterocycloalkyl, (C₁-C₆)alkylheteroaryl, (C₁-C₆)alkylaryl, (C₃-C₆)cycloalkyl, heterocycloalkyl, aryl and heteroaryl are optionally substituted by R₁₀;
Z is CH₂ or O;
q is 1-3;
n is selected from 1-4;
p and m are independently 1 or 2;
and t is selected from 0-2;
or pharmaceutically acceptable salts, metabolites, isomers, stereoisomers, atropisomers, conformers, tautomers, polymorphs, hydrates or solvates thereof.

2. The compound of formula (I) according to claim 1, wherein R₅ and R₆ is (C₁-C₆)alkyl.

3. The compound of formula (I) according to claim 1, wherein
wherein,
X is a bond or 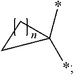
* denotes point of attachment to ring A and to the rest of molecule;
when X is a bond, ring A is a mono or bicyclic heteroaryl containing at least one N and said N is point of attachment to X;
or when X is 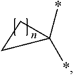
ring A is monocyclic aryl or heteroaryl;
ring B is monocyclic aryl;
R₁ and R₂ are independently selected from hydrogen, halogen, CF₃, (C₁-C₆)alkyl, (C₃-C₆)cycloalkyl, (C₂-C₅)alkynyl-R₁₃, aryl and heteroaryl; said (C₃-C₆)cycloalkyl, aryl and heteroaryl are optionally substituted by R₁₀;
R₃ and R₄ are independently selected from hydrogen, NR₁₁R₁₂ and OR₉;
R₅, R₆, R₇ and R₈ are independently selected from hydrogen and (C₁-C₆)alkyl, R₉ is independently selected from hydrogen, (C₁-C₆)alkyl and (C₃-C₆)cycloalkyl, said (C₃-C₆)cycloalkyl is optionally substituted by R₁₀;
R₁₀ is independently selected from hydrogen, oxo, OR₉, and (C₁-C₆)alkyl;
R₁₁ and R₁₂ are independently selected from hydrogen and (C₁-C₆)alkyl;
R₁₃ is independently selected from hydrogen, (C₁-C₆)alkyl-OR₉, (C₁-C₆)alkyl-N(R₉)COR₉, (C₁-C₆)alkyl-NR₁₁R₁₂, heteroaryl, (C₃-C₆)cycloalkyl, (C₁-C₆)alkyl(C₃-C₆)cycloalkyl, (C₁-C₆)alkylheterocycloalkyl; said (C₃-C₆)cycloalkyl, (C₁-C₆)alkyl(C₃-C₆)cycloalkyl, (C₁-C₆)alkylheterocycloalkyl, and heteroaryl are optionally substituted by R₁₀;
Z is CH₂ or O;
n is 1;
q is 1 to 2;
and p is 1 or 2
or pharmaceutically acceptable salts, metabolites, isomers, stereoisomers, atropisomers, conformers, tautomers, polymorphs, hydrates and solvates thereof.

4. The compound of formula (I) according to claim 1, wherein the compound is selected from the group consisting of:
2-{[4-amino-3-(7-hydroxy-2,3-dihydro-1H-inden-4-yl)-1H-pyrazolo[3,4-d]pyrimidin-1-yl]methyl}-5-chloro-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-methyl-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-chloro-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-chloro-3-(2H-indazol-2-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-chloro-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-chloro-3-(1H-pyrazol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-methyl-3-(1H-pyrazol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-methyl-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-bromo-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(pyridin-3-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(3,5-dimethyl-1H-pyrazol-1-yl)-5-methylquinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-chloro-3-(3,5-dimethyl-1H-pyrazol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-bromo-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(1H-pyrrol-1-yl)-5-(thiophen-3-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(5-methylfuran-2-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(1-phenylcyclopropyl)-5-(pyridin-3-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(5-methylfuran-2-yl)-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(pyridin-4-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-hydroxyprop-1-yn-1-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-ethynyl-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(1-phenylcyclopropyl)-5-(pyridin-4-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-541-methyl-1H-pyrazol-5-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(furan-3-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-hydroxyprop-1-yn-1-yl)-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(1-phenylcyclopropyl)-5-(thiophen-3-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-fluoro-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-6-methyl-3-(1H-pyrrol-1-yl)quinazolin-4(3H)-one;
N-{3-[2-{[4-amino-3-(7-hydroxy-2,2-dimethyl-2,3-dihydro-1-benzofuran-4-yl)-1H-pyrazolo[3,4-d]pyrimidin-1-yl]methyl}-4-oxo-3-(1H-pyrrol-1-yl)-3,4-dihydroquinazolin-5-yl]prop-2-yn-1-yl}acetamide;
N-{3-[2-{[4-amino-3-(7-hydroxy-2,2-dimethyl-2,3-dihydro-1-benzofuran-4-yl)-1H-pyrazolo[3,4-d]pyrimidin-1-yl]methyl}-4-oxo-3-(1H-pyrrol-1-yl)-3,4-dihydroquinazolin-5-yl]prop-2-yn-1-yl}cyclopropanecarboxamide;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(1-methyl-1H-pyrazol-5-yl)-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-hydroxyprop-1-yn-1-yl)-3-[1-(3-methylphenyl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-8-methyl-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-[1-(3-methylphenyl)cyclopropyl]-5-(1-methyl-1H-pyrazol-5-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-[3-(diethylamino)prop-1-yn-1-yl]-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-[3-(morpholin-4-yl)prop-1-yn-1-yl]-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-cyclopropyl-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-hydroxyprop-1-yn-1-yl)-3-[1-(4-methylphenyl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-[3-(diethylamino)prop-1-yn-1-yl]-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-methylquinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-[1-(4-methylphenyl)cyclopropyl]-5-(1-methyl-1H-pyrazol-5-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-(1-methyl-1H-pyrazol-5-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-(3-hydroxyprop-1-yn-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(cyclopropylethynyl)-3-(2, 5-dimethyl-1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-[1-methyl-1H-pyrazol-4-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-[1-ethyl-1H-pyrazol-4-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-6-methyl-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-[3-(morpholin-4-yl)prop-1-yn-1-yl]-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(cyclopropylethynyl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-hydroxy-3-methylbut-1-yn-1-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-hydroxy-3-methylbut-1-yn-1-yl)-3-[1-(4-methylphenyl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(cyclopropylethynyl)-3-[1-(4-methylphenyl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-ethyl-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-hydroxy-3-methylbut-1-yn-1-yl)-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(cyclopropylethynyl)-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-chloro-3-[1-(2-methylphenyl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-methyl-3-[1-(2-methylphenyl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-(3-(4-methylpiperazin-1-yl)prop-1-yn-1-yl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-[3-(3-oxopiperazin-1-yl)prop-1-yn-1-yl]-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-[1-(2-methylphenyl)cyclopropyl]-5-(1-methyl-1H-pyrazol-5-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-methyl-3-[1-(thiophen-2-yl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-(4-methylpiperazin-1-yl)prop-1-yn-1-yl]-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-(1-methyl-1H-pyrazol-4-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(1H-pyrrol-1-yl)-5-(trifluoromethyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-[(1-methyl-1H-imidazol-5-yl)ethynyl]-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(cyclopropylethynyl)-3-[1-(2-methylphenyl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(1-methyl-1H-pyrazol-5-yl)-3-[1-(thiophen-3-yl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-[(1-methyl-1H-imidazol-5-yl)ethynyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-(3-(3-oxopiperazin-1-yl)prop-1-yn-1-yl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(1-methyl-1H-pyrazol-4-yl)-3-[1-(thiophen-3-yl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-[1-(2-methylphenyl)cyclopropyl]-5-(morpholin-4-yl)prop-1-yn-1-yl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(1-methyl-1H-pyrrol-2-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-[3-(morpholin-4-yl)prop-1-yn-1-yl]-3-[1-(thiophen-3-yl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-cyclopentylprop-1-yn-1-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-hydroxyphenyl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(1-methyl-1H-pyrrol-2-yl)-3-[1-(thiophen-3-yl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(cyclopropylethynyl)-3-[1-(thiophen-3-yl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-(1-methyl-1H-pyrrol-2-yl)quinazolin-4(3H)-one;
2-{[4-amino-3-(7-methoxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-methylquinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-(4-methylpiperazin-1-yl)prop-1-yn-1-yl]-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-(3-(morpholin-4-yl)prop-1-yn-1-yl]quinazolin-4(3H)-one;
2-{[4-amino-3-(7-hydroxy-2,3-dihydro-1H-inden-4-yl)-1H-pyrazolo[3,4-d]pyrimidin-1-yl]methyl-5-methyl-3-[1-(2-methylphenyl)cyclopropyl]quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(3-(3-oxopiperazin-1-yl)prop-1-yn-1-yl]-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(1-phenylcyclopropyl)-5-[3-(thiomorpholin-4-yl)prop-1-yn-1-yl]quinazolin-4(3H)-one;
2-{[4-amino-3-(7-methoxy-2,2-dimethyl-2,3-dihydro-1-benzofuran-4-yl)-1H-pyrazolo[3,4-d]pyrimidin-1-yl]methyl}-5-methyl-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-(1-ethyl-1H-pyrazol-4-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2, 5-dimethyl-1H-pyrrol-1-yl)-5-(1H-pyrazol-4-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-bromo-3-(2, 5-dimethyl-1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-(1-propyl-1H-pyrazol-4-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-(1-ethyl-1H-pyrazol-4-yl)-3-(1-phenylcyclopropyl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-3-(2,5-dimethyl-1H-pyrrol-1-yl)-5-(thiophen-3-yl)quinazolin-4(3H)-one;
2-{(1S)-1-[4-amino-3-(7-hydroxy-2,2-dimethyl-2,3-dihydro-1-benzofuran-4-yl)-1H-pyrazolo[3,4-d]pyrimidin-1-yl] ethyl}-5-methyl-3-(1H-pyrrol-1-yl)quinazolin-4(3H)-one;
2-{[4-amino-3-(7-hydroxy-2,3-dihydro-1H-inden-4-yl)-1H-pyrazolo[3,4-d]pyrimidin-1-yl]methyl}-3-(2,5-dimethyl-1H-pyrrol-1-yl)-5-(1-methyl-1H-pyrazol-4-yl)quinazolin-4(3H)-one;
2-{[4-amino-3-(7-hydroxy-2,3-dihydro-1H-inden-4-yl)-1H-pyrazolo[3,4-d]pyrimidin-1-yl]methyl}-3-(2, 5-dimethyl-1H-pyrrol-1-yl)-5-(1-ethyl-1H-pyrazol-4-yl)quinazolin-4(3H)-one;
2-{(1S)-1-[4-amino-3-(7-hydroxy-2,2-dimethyl-2,3-dihydro-1-benzofuran-4-yl)-1H-pyrazolo[3,4-d]pyrimidin-1-yl]ethyl}-3-(2, 5-dimethyl-1H-pyrrol-1-yl)-5-methylquinazolin-4(3H)-one;
2-{(1RS)-1-[4-amino-3-(7-hydroxy-2,2-dimethyl-2,3-dihydro-1-benzofuran-4-yl)-1H-pyrazolo[3,4-d]pyrimidin-1-yl]ethyl}-5-methyl-3-(1H-pyrrol-1-yl)quinazolin-4(3H)-one;
2-{(1S)-1-[4-amino-3-(7-hydroxy-2,2-dimethyl-2,3-dihydro-1-benzofuran-4-yl)-1H-pyrazolo[3,4-d]pyrimidin-1-yl]ethyl}-5-methyl-3-[1-(3-methylphenyl)cyclopropyl]quinazolin-4(3H)-one;
2-{(1S)-1-[4-amino-3-(7-hydroxy-2,3-dihydro-1H-inden-4-yl)-1H-pyrazolo[3,4-d]pyrimidin-1-yl]ethyl}-5-methyl-3-(1H-pyrrol-1-yl)quinazolin-4(3H)-one;
2-{(1S)-1-[4-amino-3-(7-hydroxy-2,2-dimethyl-2,3-dihydro-1-benzofuran-4-yl)-1H-pyrazolo[3,4-d]pyrimidin-1-yl] ethyl}-5-(1-ethyl-1H-pyrazol-4-yl)-3-(1H-pyrrol-1-yl)quinazolin-4(3H)-one;
2-{[4-amino-3-(7-hydroxy-2,2-dimethyl-2,3-dihydro-1-benzofuran-4-yl)-1H-pyrazolo[3,4-d]pyrimidin-1-yl]methyl}-5-methyl-3-(1H-pyrrol-1-yl)quinazolin-4(3H)-one methanesulfonate;
4-(4-amino-1-{[5-methyl-4-oxo-3-(1H-pyrrol-1-yl)-3,4-dihydroquinazolin-2-yl]methyl-1H-pyrazolo[3,4-d]pyrimidin-3-yl)-2,2-dimethyl-2,3-dihydro-1-benzofuran-7-yl L-glucopyranosiduronic acid; and
4-(4-amino-1-{[5-methyl-4-oxo-3-(1H-pyrrol-1-yl)-3,4-dihydroquinazolin-2-yl]methyl-1H-pyrazolo[3,4-d]pyrimidin-3-yl)-2,2-dimethyl-2,3-dihydro-1-benzofuran-7-yl hydrogen sulfate;
and pharmaceutically acceptable salts thereof.

5. A pharmaceutical composition comprising a therapeutically effective amount of one or more compounds of claim 1, in association with a pharmaceutically acceptable adjuvant or carrier.

6. A method of treating allergic or non-allergic airway diseases in a mammal, the method comprising administering a therapeutically effective amount of a compound of claim 1.

7. The method according to claim 6, wherein allergic or non-allergic airway disease is selected from chronic obstructive pulmonary disease and asthma.

8. Use of a compound as claimed in claim 1 for the preparation of a medicament for treating allergic or non-allergic airway diseases.

9. Use according to claim 8, wherein allergic or non-allergic airway disease is selected from chronic obstructive pulmonary disease and asthma.

10. (canceled)

11. The method according to claim 6, wherein the mammal is a human.

12. A method of treating allergic or non-allergic airway diseases in a mammal, the method comprising administering a therapeutically effective amount of a pharmaceutical composition of claim 5.

13. The method according to claim 12, wherein allergic or non-allergic airway disease is selected from chronic obstructive pulmonary disease and asthma.

14. The method according to claim 12, wherein the mammal is a human.
